(19) United States
(12) Patent Application Publication
(10) Pub. No.: US 2023/0007462 A1
(43) Pub. Date: Jan. 5, 2023

(54) DISCOVERY SIGNAL TRANSMISSION FOR SIDELINK COMMUNICATION OVER UNLICENSED BAND

(71) Applicant: QUALCOMM INCORPORATED, San Diego, CA (US)

(72) Inventors: Jing SUN, San Diego, CA (US); Chih-Hao LIU, San Diego, CA (US); Yisheng XUE, San Diego, CA (US); Xiaoxia ZHANG, San Diego, CA (US); Ozcan OZTURK, San Diego, CA (US); Juan MONTOJO, San Diego, CA (US)

(21) Appl. No.: 17/305,314

(22) Filed: Jul. 2, 2021

(51) Int. Cl. H04W 8/00; H04W 56/00; H04W 72/04; H04W 72/02

(52) U.S. Cl. CPC H04W 8/005; H04W 56/001; H04W 72/0453; H04W 72/02

(57) ABSTRACT

Wireless communications systems and methods related to discovery signaling for sidelink communication are provided. In one aspect, a first user equipment (UE) transmits, to a second UE in a first slot and a first portion of a subband of a shared frequency band, a synchronization communication, where the synchronization communication indicates a location of a discovery signal in a second portion of the subband of the shared frequency band. The first UE also transmits, to the second UE in the first slot and in the second portion of the subband of the shared frequency band, the discovery signal.

### TECHNICAL FIELD

This application relates to wireless communication systems, and more particularly to transmitting discovery signals for sidelink communication in a shared radio frequency band (e.g., in a shared spectrum or an unlicensed spectrum) utilized by multiple network operating entities.

### INTRODUCTION

Wireless communications systems are widely deployed to provide various types of communication content such as voice, video, packet data, messaging, broadcast, and so on. These systems may be capable of supporting communication with multiple users by sharing the available system resources (e.g., time, frequency, and power). A wireless multiple-access communications system may include a number of base stations (BSs), each simultaneously supporting communications for multiple communication devices, which may be otherwise known as user equipment (UE).

To meet the growing demands for expanded mobile broadband connectivity, wireless communication technologies are advancing from the long term evolution (LTE) technology to a next generation new radio (NR) technology, which may be referred to as 5^{th} Generation (5G). For example, NR is designed to provide a lower latency, a higher bandwidth or a higher throughput, and a higher reliability than LTE. NR is designed to operate over a wide array of spectrum bands, for example, from low-frequency bands below about 1 gigahertz (GHz) and mid-frequency bands from about 1 GHz to about 6 GHz, to high-frequency bands such as millimeter wave (mmWave) bands. NR is also designed to operate across different spectrum types, from licensed spectrum to unlicensed and shared spectrum. Spectrum sharing enables operators to opportunistically aggregate spectrums to dynamically support high-bandwidth services. Spectrum sharing can extend the benefit of NR technologies to operating entities that may not have access to a licensed spectrum.

In a wireless communication network, a BS may communicate with a UE in an uplink direction and a downlink direction. Sidelink was introduced in LTE to allow a UE to send data to another UE without tunneling through the BS and/or an associated core network. The LTE sidelink technology had been extended to provision for device-to-device (D2D) communications, vehicle-to-everything (V2X) communications, and/or cellular vehicle-to-everything (C-V2X) communications. Similarly, NR may be extended to support sidelink communications for D2D, V2X, and/or C-V2X over a dedicated spectrum, a licensed spectrum, and/or an unlicensed spectrum.

### BRIEF SUMMARY OF SOME EXAMPLES

The following summarizes some aspects of the present disclosure to provide a basic understanding of the discussed technology. This summary is not an extensive overview of all contemplated features of the disclosure and is intended neither to identify key or critical elements of all aspects of the disclosure nor to delineate the scope of any or all aspects of the disclosure. Its sole purpose is to present some concepts of one or more aspects of the disclosure in summary form as a prelude to the more detailed description that is presented later.

One aspect of the present disclosure includes a method performed by a first user equipment (UE). The method includes transmitting, to a second UE in a first slot and a first portion of a subband of a shared frequency band, a synchronization communication, where the synchronization communication indicates a location of a discovery signal in a second portion of the subband of the shared frequency band; and transmitting, to the second UE in the first slot and in the second portion of the subband of the shared frequency band, the discovery signal.

One aspect of the present disclosure includes a method performed by a first user equipment (UE). The method includes receiving, from a second UE in a first slot and first portion of a subband of a shared frequency band, a synchronization communication, where the synchronization communication indicates a location of a discovery signal in a second portion of the subband of the shared frequency band; and receiving, from the second UE based on the synchronization communication, the discovery signal in the first slot and in the second portion of the subband of the shared frequency band.

One aspect of the present disclosure includes a first user equipment (UE). The first user equipment includes a transceiver; and a processor in communication with the transceiver. The processor is configured to cause the transceiver to: transmit, to a second UE in a first slot and a first portion of a subband of a shared frequency band, a synchronization communication, where the synchronization communication indicates a location of a discovery signal in a second portion of the subband of the shared frequency band; and transmit, to the second UE in the first slot and in the second portion of the subband of the shared frequency band, the discovery signal.

One aspect of the present disclosure includes a first user equipment (UE). The first user equipment includes a transceiver; and a processor in communication with the transceiver. The processor is configured to cause the transceiver to: receive, from a second UE in a first slot and first portion of a subband of a shared frequency band, a synchronization communication, where the synchronization communication indicates a location of a discovery signal in a second portion of the subband of the shared frequency band; and receive, from the second UE based on the synchronization communication, the discovery signal in the first slot and in the second portion of the subband of the shared frequency band.

Other aspects, features, and embodiments of the present invention will become apparent to those of ordinary skill in the art, upon reviewing the following description of specific, exemplary embodiments of the present invention in conjunction with the accompanying figures. While features of the present invention may be discussed relative to certain embodiments and figures below, all embodiments of the present invention can include one or more of the advantageous features discussed herein. In other words, while one or more embodiments may be discussed as having certain advantageous features, one or more of such features may also be used in accordance with the various embodiments of the invention discussed herein. In similar fashion, while exemplary embodiments may be discussed below as device, system, or method embodiments it should be understood that such exemplary embodiments can be implemented in various devices, systems, and methods.

### BRIEF DESCRIPTION OF THE DRAWINGS

FIG. 1 illustrates a wireless communication network according to some aspects of the present disclosure.

FIG. 2 illustrates a wireless communication network that provisions for sidelink communications according to some aspects of the present disclosure.

FIG. 3 illustrates a sidelink communication scheme according to some aspects of the present disclosure.

FIG. 4 illustrates a discovery signal transmission scheme in a sidelink communication scenario according to some aspects of the present disclosure.

FIG. 5 is a simplified block diagram of an exemplary frame structure of a sidelink master information block according to some aspects of the present disclosure.

FIG. 6 is a signaling diagram of a method for discovery signaling in a sidelink communication scenario according to some aspects of the present disclosure.

FIG. 7 is a block diagram of an exemplary user equipment (UE) according to some aspects of the present disclosure.

FIG. 8 is a flow diagram of a discovery signal transmission scheme in a sidelink communication scenario according to some aspects of the present disclosure.

FIG. 9 is a flow diagram of a discovery signal receiving scheme in a sidelink communication scenario according to some aspects of the present disclosure.

### DETAILED DESCRIPTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. However, it will be apparent to those skilled in the art that these concepts may be practiced without these specific details. In some instances, well-known structures and components are shown in block diagram form in order to avoid obscuring such concepts.

This disclosure relates generally to wireless communications systems, also referred to as wireless communications networks. In various embodiments, the techniques and apparatus may be used for wireless communication networks such as code division multiple access (CDMA) networks, time division multiple access (TDMA) networks, frequency division multiple access (FDMA) networks, orthogonal FDMA (OFDMA) networks, single-carrier FDMA (SC-FDMA) networks, LTE networks, Global System for Mobile Communications (GSM) networks, 5^{th} Generation (5G) or new radio (NR) networks, as well as other communications networks. As described herein, the terms “networks” and “systems” may be used interchangeably.

An OFDMA network may implement a radio technology such as evolved UTRA (E-UTRA), Institute of Electrical and Electronics Engineers (IEEE) 802.11, IEEE 802.16, IEEE 802.20, flash-OFDM and the like. UTRA, E-UTRA, and GSM are part of universal mobile telecommunication system (UMTS). In particular, long term evolution (LTE) is a release of UMTS that uses E-UTRA. UTRA, E-UTRA, GSM, UMTS and LTE are described in documents provided from an organization named “3rd Generation Partnership Project” (3GPP), and cdma2000 is described in documents from an organization named “3rd Generation Partnership Project 2” (3GPP2). These various radio technologies and standards are known or are being developed. For example, the 3rd Generation Partnership Project (3GPP) is a collaboration between groups of telecommunications associations that aims to define a globally applicable third generation (3G) mobile phone specification. 3GPP long term evolution (LTE) is a 3GPP project which was aimed at improving the UMTS mobile phone standard. The 3GPP may define specifications for the next generation of mobile networks, mobile systems, and mobile devices. The present disclosure is concerned with the evolution of wireless technologies from LTE, 4G, 5G, NR, and beyond with shared access to wireless spectrum between networks using a collection of new and different radio access technologies or radio air interfaces.

5G networks contemplate diverse deployments, diverse spectrum, and diverse services and devices that may be implemented using an OFDM-based unified, air interface. In order to achieve these goals, further enhancements to LTE and LTE-A are considered in addition to development of the new radio technology for 5G NR networks. The 5G NR will be capable of scaling to provide coverage (1) to a massive Internet of things (IoTs) with a ultra-high density (e.g., ˜1M nodes/km²), ultra-low complexity (e.g., ˜10s of bits/sec), ultra-low energy (e.g., ˜10+ years of battery life), and deep coverage with the capability to reach challenging locations; (2) including mission-critical control with strong security to safeguard sensitive personal, financial, or classified information, ultra-high reliability (e.g., ˜99.9999% reliability), ultra-low latency (e.g., ˜1 ms), and users with wide ranges of mobility or lack thereof; and (3) with enhanced mobile broadband including extreme high capacity (e.g., ˜10 Tbps/km²), extreme data rates (e.g., multi-Gbps rate, 100+ Mbps user experienced rates), and deep awareness with advanced discovery and optimizations.

The 5G NR may be implemented to use optimized OFDM-based waveforms with scalable numerology and transmission time interval (TTI); having a common, flexible framework to efficiently multiplex services and features with a dynamic, low-latency time division duplex (TDD)/frequency division duplex (FDD) design; and with advanced wireless technologies, such as massive multiple input, multiple output (MIMO), robust millimeter wave (mmWave) transmissions, advanced channel coding, and device-centric mobility. Scalability of the numerology in 5G NR, with scaling of subcarrier spacing, may efficiently address operating diverse services across diverse spectrum and diverse deployments. For example, in various outdoor and macro coverage deployments of less than 3 GHz FDD/TDD implementations, subcarrier spacing may occur with 15 kHz, for example over 5, 10, 20 MHz, and the like bandwidth (BW). For other various outdoor and small cell coverage deployments of TDD greater than 3 GHz, subcarrier spacing may occur with 30 kHz over 80/100 MHz BW. For other various indoor wideband implementations, using a TDD over the unlicensed portion of the 5 GHz band, the subcarrier spacing may occur with 60 kHz over a 160 MHz BW. Finally, for various deployments transmitting with mmWave components at a TDD of 28 GHz, subcarrier spacing may occur with 120 kHz over a 500 MHz BW.

The scalable numerology of the 5G NR facilitates scalable TTI for diverse latency and quality of service (QoS) requirements. For example, shorter TTI may be used for low latency and high reliability, while longer TTI may be used for higher spectral efficiency. The efficient multiplexing of long and short TTIs to allow transmissions to start on symbol boundaries. 5G NR also contemplates a self-contained integrated subframe design with UL/downlink scheduling information, data, and acknowledgement in the same subframe. The self-contained integrated subframe supports communications in unlicensed or contention-based shared spectrum, adaptive UL/downlink that may be flexibly configured on a per-cell basis to dynamically switch between UL and downlink to meet the current traffic needs.

Various other aspects and features of the disclosure are further described below. It should be apparent that the teachings herein may be embodied in a wide variety of forms and that any specific structure, function, or both being disclosed herein is merely representative and not limiting. Based on the teachings herein one of an ordinary level of skill in the art should appreciate that an aspect disclosed herein may be implemented independently of any other aspects and that two or more of these aspects may be combined in various ways. For example, an apparatus may be implemented, or a method may be practiced using any number of the aspects set forth herein. In addition, such an apparatus may be implemented, or such a method may be practiced using other structure, functionality, or structure and functionality in addition to or other than one or more of the aspects set forth herein. For example, a method may be implemented as part of a system, device, apparatus, and/or as instructions stored on a computer readable medium for execution on a processor or computer. Furthermore, an aspect may comprise at least one element of a claim.

Sidelink communications refer to the communications among user equipment devices (UEs) without tunneling through a base station (BS) and/or a core network. Sidelink communications can be communicated over a physical sidelink control channel (PSCCH) and a physical sidelink shared channel (PSSCH). The PSCCH and PSSCH are analogous to a physical downlink control channel (PDCCH) and a physical downlink shared channel (PDSCH) in downlink (DL) communication between a BS and a UE. For instance, the PSCCH may carry sidelink control information (SCI) and the PSSCH may carry sidelink data (e.g., user data). Each PSCCH is associated with a corresponding PSSCH, where SCI in a PSCCH may carry reservation and/or scheduling information for sidelink data transmission in the associated PSSCH. Use cases for sidelink communication may include V2X, enhanced mobile broadband (eMBB), industrial IoT (IIoT), and/or NR-lite.

As used herein, the term “sidelink UE” can refer to a user equipment device performing a device-to-device communication or other types of communications with another user equipment device independent of any tunneling through the BS (e.g., gNB) and/or an associated core network. As used herein, the term “sidelink transmitting UE” can refer to a user equipment device performing a sidelink transmission operation. As used herein, the term “sidelink receiving UE” can refer to a user equipment device performing a sidelink reception operation. As used herein, the terms “anchor UE” or “sidelink anchor UE” refer to a sidelink UE designated as an anchor node with a stand-alone sidelink configuration that can initiate sidelink operations autonomously (e.g., independent of any cell and/or associated core network), and the terms are interchangeable without departing from the scope of the present disclosure.

The deployment of NR over an unlicensed spectrum is referred to as NR-unlicensed (NR-U). Some studies have been conducted for NR-U deployment over 5 gigahertz (GHz) unlicensed bands. Federal Communications Commission (FCC) and European Telecommunications Standards Institute (ETSI) are working on regulating 6 GHz as a new unlicensed band for wireless communications. The addition of 6 GHz bands allows for hundreds of megahertz (MHz) of bandwidth (BW) available for unlicensed band communications. Additionally, NR-U can also be deployed over 2.4 GHz unlicensed bands, which are currently shared by various radio access technologies (RATs), such as IEEE 802.11 wireless local area network (WLAN) or WiFi and/or license assisted access (LAA). Sidelink can benefit from utilizing the additional bandwidth available in an unlicensed spectrum. However, channel access in a certain unlicensed spectrum may be regulated by authorities. For instance, regulations in a 2.4 GHz band allows a node to transmit without performing an LBT when the node applies frequency-hopping to transmissions and satisfies a transmission sequence or on/off pattern with a maximum transmission duration of about 5 ms and a minimum silent or gap duration of about 5 ms between transmissions.

NR supports two modes of radio resource allocations (RRA), a mode-1 RRA and a mode-2 RRA, for sidelink over a licensed spectrum. The mode-1 RRA supports network controlled RRA that can be used for in-coverage sidelink communication. For instance, a serving BS (e.g., gNB) may determine a radio resource on behalf of a sidelink UE and transmit an indication of the radio resource to the sidelink UE. In some aspects, the serving BS grants a sidelink transmission with downlink control information (DCI). For this mode, however, there is significant base station involvement and is only operable when the sidelink UE is within the coverage area of the serving BS. The mode-2 RRA supports autonomous RRA that can be used for out-of-coverage sidelink UEs or partial-coverage sidelink UEs. For instance, an out-of-coverage sidelink UE or a partial-coverage UE may be preconfigured with a sidelink resource pool and may select a radio resource from the preconfigured sidelink resource pool for sidelink communication. For this mode, it may be possible for V2X systems, CV2X systems, or other sidelink communication systems to operate independent of the serving BS. However, the mode-2 RRA relies on sidelink settings across different UEs and/or environments. Accordingly, for mode-2 RRA the sidelink settings may be coordinated so that each sidelink UE can communicate with one another. As a result, UE vendors (e.g., different automotive manufacturers or other UE manufacturers) may need to coordinate and implement common sidelink settings. This may pose a substantial burden on the UE vendors to develop and implement uniform sidelink settings so that all NR-U sidelink UE devices can communicate via respective sidelink connections. As such, the present disclosure describes methods, devices, and systems for facilitating the deployment of an NR-U sidelink system as a stand-alone system.

For example, the present application describes mechanisms for transmitting and receiving discovery signals in a sidelink communication scenario. The mechanisms described in this application allow for the transmission and reception of a discovery signal integrated with a synchronization signal, such as a synchronization signal block (SSB). In particular, a first sidelink UE may transmit, to a second sidelink UE, a discovery signal that is aligned in time with the SSB, and that occupies a plurality of resource blocks (RBs) in the subband that is contiguous with the RBs used to transmit the synchronization signal. Further, the first sidelink UE may transmit the discovery signal such that it is quasi co-located (QCL) with the synchronization signal. The discovery signal may include configuration information associated with the sidelink communication scheme between the UEs, such as resource pool configurations, UE identity, a UE's network service information, and/or any other suitable sidelink configuration information. In some aspects, the structure of the discovery signal may be similar or identical to the structure of a remaining minimum system information (RMSI).

Aspects of the present disclosure provide several benefits. For example, because the mechanisms described herein use time/frequency resources (e.g., RBs) that are associated with a configured synchronization signal, the discovery signal indication schemes advantageously allow for the transmission and reception of sidelink discovery information such that UEs from a variety of manufacturers and/or operators can receive the discovery information without additional cooperation or agreement for specific discovery signal settings and transmission structures. Further, transmitting the discovery signal in an otherwise unused portion of the subband in which the synchronization signal is transmitted makes efficient use of network resources, improving throughput, reducing power consumption of the UE devices, and increasing user satisfaction with the wireless network. Further, control information and/or parameters for decoding and using the discovery information can be indicated in system information carried by the synchronization signal, such as a master information block (MIB) carried in the physical broadcast channel (PBCH) of a SSB. In this way, more of the RBs in the subband that are not designated for the synchronization communication can be used to transmit the discovery signal, further improving the efficiency of the sidelink communications, improving throughput, reducing power consumption, and increasing user satisfaction with the wireless network.

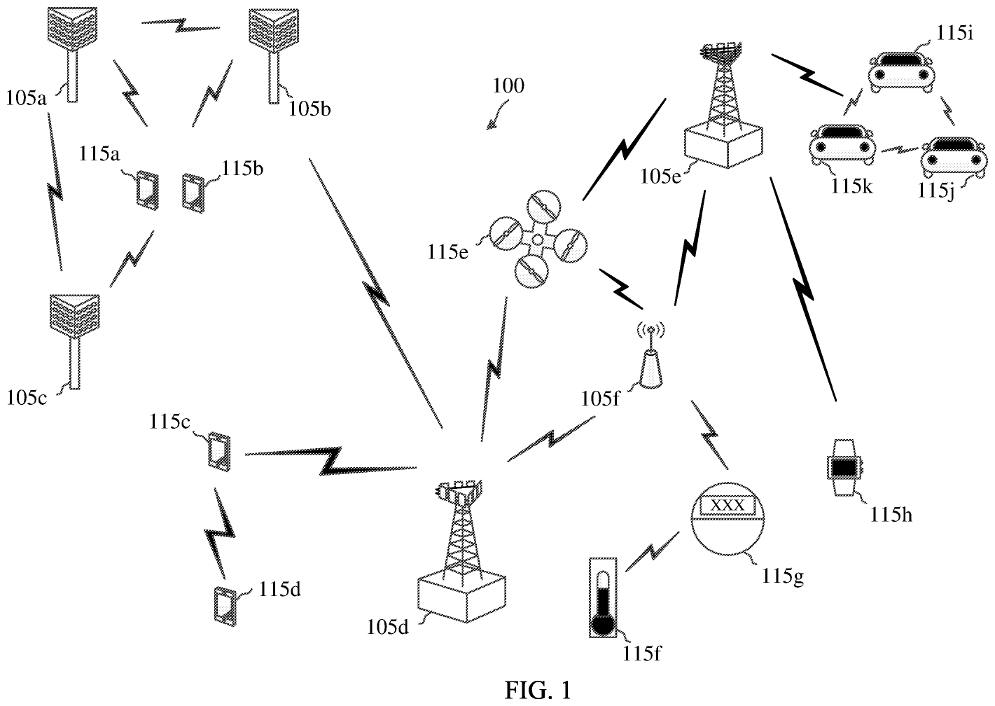

FIG. 1 illustrates a wireless communication network 100 according to some aspects of the present disclosure. The network 100 may be a 5G network. The network 100 includes a number of base stations (BSs) 105 (individually labeled as 105a, 105b, 105c, 105d, 105e, and 105f) and other network entities. A BS 105 may be a station that communicates with UEs 115 and may also be referred to as an evolved node B (eNB), a next generation eNB (gNB), an access point, and the like. Each BS 105 may provide communication coverage for a particular geographic area. In 3GPP, the term “cell” can refer to this particular geographic coverage area of a BS 105 and/or a BS subsystem serving the coverage area, depending on the context in which the term is used.

A BS 105 may provide communication coverage for a macro cell or a small cell, such as a pico cell or a femto cell, and/or other types of cell. A macro cell generally covers a relatively large geographic area (e.g., several kilometers in radius) and may allow unrestricted access by UEs with service subscriptions with the network provider. A small cell, such as a pico cell, would generally cover a relatively smaller geographic area and may allow unrestricted access by UEs with service subscriptions with the network provider. A small cell, such as a femto cell, would also generally cover a relatively small geographic area (e.g., a home) and, in addition to unrestricted access, may also provide restricted access by UEs having an association with the femto cell (e.g., UEs in a closed subscriber group (CSG), UEs for users in the home, and the like). A BS for a macro cell may be referred to as a macro BS. A BS for a small cell may be referred to as a small cell BS, a pico BS, a femto BS or a home BS. In the example shown in FIG. 1, the BSs 105d and 105e may be regular macro BSs, while the BSs 105a-105c may be macro BSs enabled with one of three dimension (3D), full dimension (FD), or massive MIMO. The BSs 105a-105c may take advantage of their higher dimension MIMO capabilities to exploit 3D beamforming in both elevation and azimuth beamforming to increase coverage and capacity. The BS 105f may be a small cell BS which may be a home node or portable access point. A BS 105 may support one or multiple (e.g., two, three, four, and the like) cells.

The network 100 may support synchronous or asynchronous operation. For synchronous operation, the BSs may have similar frame timing, and transmissions from different BSs may be approximately aligned in time. For asynchronous operation, the BSs may have different frame timing, and transmissions from different BSs may not be aligned in time.

The UEs 115 are dispersed throughout the wireless network 100, and each UE 115 may be stationary or mobile. A UE 115 may also be referred to as a terminal, a mobile station, a subscriber unit, a station, or the like. A UE 115 may be a cellular phone, a personal digital assistant (PDA), a wireless modem, a wireless communication device, a handheld device, a tablet computer, a laptop computer, a cordless phone, a wireless local loop (WLL) station, or the like. In one aspect, a UE 115 may be a device that includes a Universal Integrated Circuit Card (UICC). In another aspect, a UE may be a device that does not include a UICC. In some aspects, the UEs 115 that do not include UICCs may also be referred to as IoT devices or internet of everything (IoE) devices. The UEs 115a-115d are examples of mobile smart phone-type devices accessing network 100. A UE 115 may also be a machine specifically configured for connected communication, including machine type communication (MTC), enhanced MTC (eMTC), narrowband IoT (NB-IoT) and the like. The UEs 115e-115h are examples of various machines configured for communication that access the network 100. The UEs 115i-115k are examples of vehicles equipped with wireless communication devices configured for communication that access the network 100. A UE 115 may be able to communicate with any type of the BSs, whether macro BS, small cell, or the like. In FIG. 1, a lightning bolt (e.g., communication links) indicates wireless transmissions between a UE 115 and a serving BS 105, which is a BS designated to serve the UE 115 on the downlink (DL) and/or uplink (UL), desired transmission between BSs 105, backhaul transmissions between BSs, or sidelink transmissions between UEs 115.

In operation, the BSs 105a-105c may serve the UEs 115a and 115b using 3D beamforming and coordinated spatial techniques, such as coordinated multipoint (CoMP) or multi-connectivity. The macro BS 105d may perform backhaul communications with the BSs 105a-105c, as well as small cell, the BS 105f. The macro BS 105d may also transmits multicast services which are subscribed to and received by the UEs 115c and 115d. Such multicast services may include mobile television or stream video, or may include other services for providing community information, such as weather emergencies or alerts, such as Amber alerts or gray alerts.

The BSs 105 may also communicate with a core network. The core network may provide user authentication, access authorization, tracking, Internet Protocol (IP) connectivity, and other access, routing, or mobility functions. At least some of the BSs 105 (e.g., which may be an example of a gNB or an access node controller (ANC)) may interface with the core network through backhaul links (e.g., NG-C, NG-U, etc.) and may perform radio configuration and scheduling for communication with the UEs 115. In various examples, the BSs 105 may communicate, either directly or indirectly (e.g., through core network), with each other over backhaul links (e.g., X1, X2, etc.), which may be wired or wireless communication links.

The network 100 may also support mission critical communications with ultra-reliable and redundant links for mission critical devices, such as the UE 115e, which may be a drone. Redundant communication links with the UE 115e may include links from the macro BSs 105d and 105e, as well as links from the small cell BS 105f. Other machine type devices, such as the UE 115f (e.g., a thermometer), the UE 115g (e.g., smart meter), and UE 115h (e.g., wearable device) may communicate through the network 100 either directly with BSs, such as the small cell BS 105f, and the macro BS 105e, or in multi-step-size configurations by communicating with another user device which relays its information to the network, such as the UE 115f communicating temperature measurement information to the smart meter, the UE 115g, which is then reported to the network through the small cell BS 105f. The network 100 may also provide additional network efficiency through dynamic, low-latency TDD/FDD communications, such as V2V, V2X, C-V2X communications between a UE 115i, 115j, or 115k and other UEs 115, and/or vehicle-to-infrastructure (V2I) communications between a UE 115i, 115j, or 115k and a BS 105.

In some implementations, the network 100 utilizes OFDM-based waveforms for communications. An OFDM-based system may partition the system BW into multiple (K) orthogonal subcarriers, which are also commonly referred to as subcarriers, tones, bins, or the like. Each subcarrier may be modulated with data. In some instances, the subcarrier spacing between adjacent subcarriers may be fixed, and the total number of subcarriers (K) may be dependent on the system BW. The system BW may also be partitioned into subbands. In other instances, the subcarrier spacing and/or the duration of TTIs may be scalable.

In some aspects, the BSs 105 can assign or schedule transmission resources (e.g., in the form of time-frequency resource blocks (RB)) for downlink (DL) and uplink (UL) transmissions in the network 100. DL refers to the transmission direction from a BS 105 to a UE 115, whereas UL refers to the transmission direction from a UE 115 to a BS 105. The communication can be in the form of radio frames. A radio frame may be divided into a plurality of subframes or slots, for example, about 10. Each slot may be further divided into mini-slots. In a FDD mode, simultaneous UL and DL transmissions may occur in different frequency bands. For example, each subframe includes a UL subframe in a UL frequency band and a DL subframe in a DL frequency band. In a TDD mode, UL and DL transmissions occur at different time periods using the same frequency band. For example, a subset of the subframes (e.g., DL subframes) in a radio frame may be used for DL transmissions and another subset of the subframes (e.g., UL subframes) in the radio frame may be used for UL transmissions.

The DL subframes and the UL subframes can be further divided into several regions. For example, each DL or UL subframe may have pre-defined regions for transmissions of reference signals, control information, and data. Reference signals are predetermined signals that facilitate the communications between the BSs 105 and the UEs 115. For example, a reference signal can have a particular pilot pattern or structure, where pilot tones may span across an operational BW or frequency band, each positioned at a pre-defined time and a pre-defined frequency. For example, a BS 105 may transmit cell specific reference signals (CRSs) and/or channel state information—reference signals (CSI-RSs) to enable a UE 115 to estimate a DL channel. Similarly, a UE 115 may transmit sounding reference signals (SRSs) to enable a BS 105 to estimate a UL channel Control information may include resource assignments and protocol controls. Data may include protocol data and/or operational data. In some aspects, the BSs 105 and the UEs 115 may communicate using self-contained subframes. A self-contained subframe may include a portion for DL communication and a portion for UL communication. A self-contained subframe can be DL-centric or UL-centric. A DL-centric subframe may include a longer duration for DL communication than for UL communication. A UL-centric subframe may include a longer duration for UL communication than for UL communication.

In some aspects, the network 100 may be an NR network deployed over a licensed spectrum. The BSs 105 can transmit synchronization signals (e.g., including a primary synchronization signal (PSS) and a secondary synchronization signal (SSS)) in the network 100 to facilitate synchronization. The BSs 105 can broadcast system information associated with the network 100 (e.g., including a master information block (MIB), remaining minimum system information (e.g., RMSI), and other system information (OSI)) to facilitate initial network access. In some instances, the BSs 105 may broadcast the PSS, the SSS, and/or the MIB in the form of synchronization signal block (SSBs) over a physical broadcast channel (PBCH) and may broadcast the RMSI and/or the OSI over a physical downlink shared channel (e.g., PDSCH).

In some aspects, a UE 115 attempting to access the network 100 may perform an initial cell search by detecting a PSS from a BS 105. The PSS may enable synchronization of period timing and may indicate a physical layer identity value. The UE 115 may then receive a SSS. The SSS may enable radio frame synchronization, and may provide a cell identity value. The cell identity value may be combined with the physical layer identity value to identify the cell. The PSS and the SSS may be located in a central portion of a carrier or any suitable frequencies within the carrier.

After receiving the PSS and SSS, the UE 115 may receive a MIB. The MIB may include system information for initial network access and scheduling information for RMSI and/or OSI. After decoding the MIB, the UE 115 may receive RMSI and/or OSI. The RMSI and/or OSI may include radio resource control (RRC) information related to random access channel (RACH) procedures, paging, control resource set (CORESET) for physical downlink control channel (PDCCH) monitoring, physical UL control channel (PUCCH), physical UL shared channel (PUSCH), power control, and SRS.

After obtaining the MIB, the RMSI and/or the OSI, the UE 115 can perform a random access procedure to establish a connection with the BS 105. In some examples, the random access procedure may be a four-step random access procedure. For example, the UE 115 may transmit a random access preamble and the BS 105 may respond with a random access response. The random access response (RAR) may include a detected random access preamble identifier (ID) corresponding to the random access preamble, timing advance (TA) information, a UL grant, a temporary cell-radio network temporary identifier (C-RNTI), and/or a backoff indicator. Upon receiving the random access response, the UE 115 may transmit a connection request to the BS 105 and the BS 105 may respond with a connection response. The connection response may indicate a contention resolution. In some examples, the random access preamble, the RAR, the connection request, and the connection response can be referred to as message 1 (MSG1), message 2 (MSG2), message 3 (MSG3), and message 4 (MSG4), respectively. In some examples, the random access procedure may be a two-step random access procedure, where the UE 115 may transmit a random access preamble and a connection request in a single transmission and the BS 105 may respond by transmitting a random access response and a connection response in a single transmission.

After establishing a connection, the UE 115 and the BS 105 can enter a normal operation stage, where operational data may be exchanged. For example, the BS 105 may schedule the UE 115 for UL and/or DL communications. The BS 105 may transmit UL and/or DL scheduling grants to the UE 115 via a PDCCH. The scheduling grants may be transmitted in the form of DL control information (DCI). The BS 105 may transmit a DL communication signal (e.g., carrying data) to the UE 115 via a PDSCH according to a DL scheduling grant. The UE 115 may transmit a UL communication signal to the BS 105 via a PUSCH and/or PUCCH according to a UL scheduling grant.

In some aspects, the BS 105 may communicate with a UE 115 using HARQ techniques to improve communication reliability, for example, to provide a URLLC service. The BS 105 may schedule a UE 115 for a PDSCH communication by transmitting a DL grant in a PDCCH. The BS 105 may transmit a DL data packet to the UE 115 according to the schedule in the PDSCH. The DL data packet may be transmitted in the form of a transport block (TB). If the UE 115 receives the DL data packet successfully, the UE 115 may transmit a HARQ ACK to the BS 105. Conversely, if the UE 115 fails to receive the DL transmission successfully, the UE 115 may transmit a HARQ NACK to the BS 105. Upon receiving a HARQ NACK from the UE 115, the BS 105 may retransmit the DL data packet to the UE 115. The retransmission may include the same coded version of DL data as the initial transmission. Alternatively, the retransmission may include a different coded version of the DL data than the initial transmission. The UE 115 may apply soft-combining to combine the encoded data received from the initial transmission and the retransmission for decoding. The BS 105 and the UE 115 may also apply HARQ for UL communications using substantially similar mechanisms as the DL HARQ.

In some aspects, the network 100 may operate over a system BW or a component carrier (CC) BW. The network 100 may partition the system BW into multiple BWPs (e.g., portions). A BS 105 may dynamically assign a UE 115 to operate over a certain BWP (e.g., a certain portion of the system BW). The assigned BWP may be referred to as the active BWP. The UE 115 may monitor the active BWP for signaling information from the BS 105. The BS 105 may schedule the UE 115 for UL or DL communications in the active BWP. In some aspects, a BS 105 may assign a pair of BWPs within the CC to a UE 115 for UL and DL communications. For example, the BWP pair may include one BWP for UL communications and one BWP for DL communications.

In some aspects, the network 100 may operate over a shared channel, which may include shared frequency bands and/or unlicensed frequency bands. For example, the network 100 may be an NR-U network operating over an unlicensed frequency band. In such an aspect, the BSs 105 and the UEs 115 may be operated by multiple network operating entities. To avoid collisions, the BSs 105 and the UEs 115 may employ a listen-before-talk (LBT) procedure to monitor for transmission opportunities (TXOPs) in the shared channel A TXOP may also be referred to as COT. For example, a transmitting node (e.g., a BS 105 or a UE 115) may perform an LBT prior to transmitting in the channel. When the LBT passes, the transmitting node may proceed with the transmission. When the LBT fails, the transmitting node may refrain from transmitting in the channel.

An LBT can be based on energy detection (ED) or signal detection. For an energy detection-based LBT, the LBT results in a pass when signal energy measured from the channel is below a threshold. Conversely, the LBT results in a failure when signal energy measured from the channel exceeds the threshold. For a signal detection-based LBT, the LBT results in a pass when a channel reservation signal (e.g., a predetermined preamble signal) is not detected in the channel Additionally, an LBT may be in a variety of modes. An LBT mode may be, for example, a category 4 (CAT4) LBT, a category 2 (CAT2) LBT, or a category 1 (CAT1) LBT. A CAT1 LBT is referred to a no LBT mode, where no LBT is to be performed prior to a transmission. A CAT2 LBT refers to an LBT without a random backoff period. For instance, a transmitting node may determine a channel measurement in a time interval and determine whether the channel is available or not based on a comparison of the channel measurement against a ED threshold. A CAT4 LBT refers to an LBT with a random backoff and a variable contention window (CW). For instance, a transmitting node may draw a random number and backoff for a duration based on the drawn random number in a certain time unit.

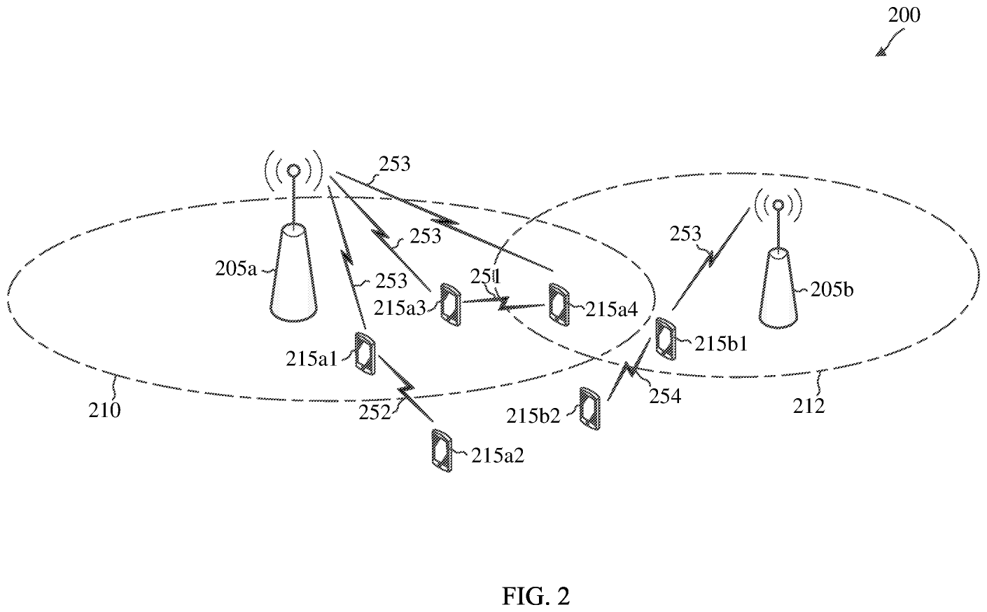

In some aspects, the network 100 may support sidelink communication among the UEs 115 over a shared radio frequency band (e.g., in a shared spectrum or an unlicensed spectrum). In some aspects, the UEs 115 may communicate with each other over a 2.4 GHz unlicensed band. The unlicensed band may be shared by multiple network operating entities using various radio access technologies (RATs) such as NR-U, WiFi, and/or licensed-assisted access (LAA) as shown in FIG. 2.

In some aspects, the network 100 may support stand-alone sidelink communication among the UEs 115 over a shared radio frequency band, in which a subset of the UEs 115 are adapted as anchor nodes (e.g., sidelink anchor UEs) and autonomously initiate sidelink operation for the UEs 115. In this respect, the sidelink anchor UEs are autonomous and can perform sidelink operations independent of any cell, such as BSs 105.

FIG. 2 illustrates an example of a wireless communication network 200 that provisions for sidelink communications according to embodiments of the present disclosure. The network 200 may correspond to a portion of the network 100. FIG. 2 illustrates two BSs 205 (shown as 205a and 205b) and six UEs 215 (shown as 215a1, 215a2, 215a3, 215a4, 215b1, and 215b2) for purposes of simplicity of discussion, though it will be recognized that embodiments of the present disclosure may scale to any suitable number of UEs 215 (e.g., the about 2, 3, 4, 5, 7 or more) and/or BSs 205 (e.g., the about 1, 3 or more). The BS 205 and the UEs 215 may be similar to the BSs 105 and the UEs 115, respectively. The BSs 205 and the UEs 215 may share the same radio frequency band for communications. In some instances, the radio frequency band may be a 2.4 GHz unlicensed band, a 5 GHz unlicensed band, or a 6 GHz unlicensed band. In general, the shared radio frequency band may be at any suitable frequency.

The BS 205a and the UEs 215a1-215a4 may be operated by a first network operating entity. The BS 205b and the UEs 215b1-215b2 may be operated by a second network operating entity. In some aspects, the first network operating entity may utilize a same RAT as the second network operating entity. For instance, the BS 205a and the UEs 215a1-215a4 of the first network operating entity and the BS 205b and the UEs 215b1-215b2 of the second network operating entity are NR-U devices. In some other aspects, the first network operating entity may utilize a different RAT than the second network operating entity. For instance, the BS 205a and the UEs 215a1-215a4 of the first network operating entity may utilize NR-U technology while the BS 205b and the UEs 215b1-215b2 of the second network operating entity may utilize WiFi or LAA technology.

In the network 200, some of the UEs 215a1-215a4 may communicate with each other in peer-to-peer communications. For example, the UE 215a1 may communicate with the UE 215a2 over a sidelink 252, the UE 215a3 may communicate with the UE 215a4 over another sidelink 251, and the UE 215b1 may communicate with the UE 215b2 over yet another sidelink 254. The sidelinks 251, 252, and 254 are unicast bidirectional links. Some of the UEs 215 may also communicate with the BS 205a or the BS 205b in a UL direction and/or a DL direction via communication links 253. For instance, the UE 215a1, 215a3, and 215a4 are within a coverage area 210 of the BS 205a, and thus may be in communication with the BS 205a. The UE 215a2 is outside the coverage area 210, and thus may not be in direct communication with the BS 205a. In some instances, the UE 215a1 may operate as a relay for the UE 215a2 to reach the BS 205a. Similarly, the UE 215b1 is within a coverage area 212 of the BS 205b, and thus may be in communication with the BS 205b and may operate as a relay for the UE 215b2 to reach the BS 205b. In some aspects, some of the UEs 215 are associated with vehicles (e.g., similar to the UEs 115i-k) and the communications over the sidelinks 251, 252, and 254 may be C-V2X communications. C-V2X communications may refer to communications between vehicles and any other wireless communication devices in a cellular network.

As discussed above, NR supports a stand-alone sidelink communication mechanism. In some aspects, a first user equipment (UE) includes a processor configured to determine system parameter information to initiate a sidelink communication, and a transceiver configured to transmit, in one or more first subbands of a plurality of subbands within a shared radio frequency during a first time period, the system parameter information, and communicate, with a second UE in a second subband of the plurality of subbands during a second time period different from the first time period, sidelink data based on the system parameter information.

For example, UE 215a2 may serve as the sidelink anchor UE and UE 215a1 may serve as a sidelink receiving UE, where UE 215a2 transmits system parameter information including timing synchronization signals over a sidelink broadcast channel (e.g., PSBCH) such that the UE 215a1 can receive and recover resource allocation and timing information to facilitate a sidelink communication with the UE 215a2. For purposes of explanation and brevity of discussion, the remaining description for FIG. 2 will be discussed in reference to UE 215a1 (e.g., sidelink receiving UE) and UE 215a2 (e.g., sidelink anchor UE).

Sidelink discovery of other sidelink transmitting UEs, such as other anchor nodes, can be facilitated through the use of a transport channel referred to as a transport sidelink discovery channel (SL-DCH), and its physical counterpart, the physical sidelink discovery channel (e.g., PSDCH). In some aspects, a sidelink transmitting UE can transmit one or more announcement messages that are generated using physical layer transport blocks with zero media access control overhead. For example, the UE 215a2 can broadcast an announcement message over the PSDCH to announce its status as an anchor node.

In various embodiments, the sidelink anchor UE may utilize the sidelink discovery procedure to: 1) announce its presence as the anchor UE to potentially proximal sidelink UEs by transmitting a message containing its application information or other useful information fields (e.g., GPS coordinates, time, and the like), and 2) monitor the presence of other proximal sidelink UEs by detecting and decoding the corresponding discovery messages, and respond to the sidelink transmitting UEs using similar discovery messages. In some instances, the discovery message may include information about the type of discovery being performed and/or the type of content (e.g., announcement, query) provided by the sidelink transmitting UE. For example, the UE 215a2 may broadcast a discovery message over the PSDCH, in which the discovery message includes an indication that the discovery message pertains to an announcement of its anchor node status.

In some aspects, UE 215a2 may perform a sensing operation on one or more of a discovery channel, such as the PSDCH, or a sidelink broadcast channel, such as the PSBCH, depending on implementation. If the UE 215a2 does not detect an existing anchor UE on the discovery channel, then the UE 215a2 may configure itself as an anchor UE and broadcast an announcement indicating itself to be the anchor UE. If the UE 215a2 detects an existing anchor UE, the UE 215a2 may determine whether there is a need for it to become an anchor node within the wireless communication network 200 or not.

In some instances, there may be multiple anchor nodes in the wireless communication network 200. A sidelink anchor UE, such as UE 215a2, may perform sensing operations on the sidelink discovery channel. In some aspects, when two anchor UEs sense each other, one anchor UE can adopt the system parameters of the other anchor UE to maintain local consistency of system parameters in the wireless communication network 200. In some instances, an in-coverage anchor UE (e.g., a sidelink anchor UE within the coverage area of an existing cell) may have priority in determining the system parameters. For example, the UE 215a1 may be configured as an anchor node such that the UE 215a2 sensed the UE 215a2 as an anchor node and determines that it is an in-coverage anchor UE based on its location being within a coverage area of BS 205a. In some embodiments, the anchor UE that adopted the new system parameters may broadcast its updated system parameters to other sidelink receiving UE over the sidelink broadcast channel. For example, the UE 215a2 may adopt the system parameters of the UE 215a1, and in turn, the UE 215a2 may broadcast its updated system parameters to other neighboring sidelink receiving UEs (e.g., 215a3, 215a4, 215b2). In other embodiments, the sidelink anchor UE may actively communicate with other sidelink receiving UEs via unicast or multicast transmissions to indicate the change in system parameters.

For in-coverage sidelink operation, where both transmitting and sidelink receiving UEs reside in the same coverage area of a BS, time synchronization is provided by the BS and there may be no need for the UE 215a2 to become an anchor UE to initiate sidelink operations by performing sidelink-specific synchronization. However, there may be several scenarios where there may be a need for the UE 215a2 to become an anchor UE to perform sidelink-specific operations: (i) in multi-cell in-coverage, where the sidelink receiving UE resides in a different asynchronous cell with respect to the sidelink transmitting UE; (ii) in partial-coverage, where the sidelink receiving UE is out of coverage and may need to acquire synchronization from the in-coverage sidelink transmitting UE; and/or (iii) out of coverage, where both sidelink UEs are outside the coverage of a cell and the sidelink transmitting UE decides to act as a reference synchronization source (referred to as the anchor UE).

In some aspects, the UE 215a2 may evaluate other factors, including but not limited to, a transmission priority level of the UE 215a2, an application type of the UE 215a2, number of sidelink UE participants within synchronization range of the UE 215a2, and/or a network congestion level within a sidelink coverage area of the UE 215a2.

In some aspects, if the UE 215a2 determines that one or more of the above-mentioned factors are satisfied through a quantitative and/or qualitative analysis, the UE 215a2 may determine that it can become an anchor node. In this respect, the UE 215a2 can adopt the system parameters and any associated timing parameters of the detected anchor UE (e.g., UE 215a1). In some aspects, the detected anchor UE may propagate its system parameters to the UE 215a2. This would allow multiple anchor UEs to coexist in the network 200 while the anchor UEs have corresponding system parameters to maintain local consistency of the sidelink system operation.

In various embodiments, the UE 215a2 as the anchor node may autonomously form mode-specific time and frequency radio resource pools. The UE 215a2 may allocate specific resources for control and data from these radio resource pools to other sidelink receiving UEs. In some instances, the UE 215a2 may form a radio resource pool for discovery communication (hereinafter referred to as “sidelink discovery resource pool”). In other instances, the UE 215a2 may form radio resource pools for control and data communications, such as a control channel resource pool (hereinafter referred to as “PSCCH resource pool”) and a data channel resource pool (hereinafter referred to as “PSSCH resource pool”). In various embodiments, the UE 215a2 may provide a transmission resource pool configuration that includes configuration information for a discovery resource pool configuration and a control/data communication resource pool configuration.

Sidelink receiving UEs (e.g., UE 215a1) may monitor multiple resources to listen for discovery announcements communicated by anchor UEs (e.g., UE 215a2) to minimize and/or avoid sidelink UE interference. In some embodiments, the UE 215a2 may autonomously determine a sidelink discovery resource pool that contains certain subframes that carry sidelink control signals, whereas the remainder of subframes may carry the sidelink data. In this respect, sidelink receiving UEs may be assigned time and frequency resources for sending and/or monitoring discovery messages to other sidelink receiving UEs from the sidelink discovery resource pool. In selecting the resources from the pool, the UE 215a2 may attempt to avoid the assignment of common time/frequency resources to different sidelink receiving UEs. In some embodiments, the UE 215a2 may select the time and frequency resources from the resource pool using a randomization parameter to minimize (or at least reduce) the number of resource allocation conflicts.

In some embodiments, the discovery resource pool configuration may indicate which RBs are available for discovery transmissions, whether broadcast synchronization signals can be triggered in response to the discovery message, whether such broadcast synchronization signals are to be transmitted at once or periodically, and/or indication of how sidelink radio resources can be allocated to different discovery transmissions (e.g., autonomously by the sidelink anchor UE or sidelink transmitting UE). The discovery resource pool configuration may include additional parameter information that indicates which resources the sidelink receiving UE can monitor for identifying potential discovery announcement messages and other parameter information used for tuning channel estimation and channel decoding operations at the sidelink receiving UE. In operation of the discovery mode, the discovery messages may follow transmission of the broadcast synchronization signals in accordance with the time/frequency resource allocations defined in the discovery resource pool configuration.

In a stand-alone sidelink communication, the radio resource pools for PSCCH and PSSCH may be separate. In some instances, the PSSCH radio resource pool may begin at a fixed time-offset with respect to the PSCCH radio resource pool. In some embodiments, the UE 215a2 as the anchor node may autonomously select a time/frequency resource from the PSCCH radio resource pool based on a randomization parameter to allocate resources for the sidelink control channel, PSCCH. The UE 215a2 may also autonomously select a time/frequency resource from the PSSCH radio resource pool based on a UE-specific subframe bitmap to allocate resources for the sidelink shared channel, PSSCH. In various embodiments, sidelink communication between sidelink UEs can be facilitated through the use of a transport channel, transport sidelink shared channel (SL-SCH), and its physical counterpart, the PSSCH.

The timing synchronization and system information acquisition by sidelink receiving UEs (e.g., 215a1) may be facilitated by a sidelink broadcast transport channel, SL-BCH, and its physical counterpart, PSBCH. These channels may be used for broadcasting a set of preambles and system parameter information within proximity of the UE 215a2. The set of primary and secondary preambles, PSS and SSS, can be used for synchronization of the sidelink receiving UEs (e.g., 215a1). As described herein, the sidelink master information block, SL-MIB, can carry the sidelink system parameter information. Through the acquisition of the PSS/SSS preambles, a proximal sidelink receiving UE (e.g., UE 215a1) may acquire time synchronization with the sidelink anchor UE (e.g., UE 215a2) and obtain its physical identity. The SL-MIB may include system information for initial network access and scheduling information for the RMSI. The SL-MIB may also include one or more predefined sets of initial BWP configurations and/or an initial transmission resource pool configuration. In some aspects, the S-SSB is transmitted within the bandwidth defined in an initial BWP configuration.

After decoding the SL-MIB, the sidelink receiving UE (e.g., UE 215a1) may recover the RMSI based on a pointer included in a repurposed bit field of the SL-MIB. The RMSI may include additional system parameters. In some aspects, the RMSI includes intra-cell guard band information for NR-U systems for use by the sidelink receiving UE (e.g., UE 215a1) to derive resource block sets. In various embodiments, the RMSI includes transmit resource pool configuration information and/or receive resource pool configuration information. The transmit resource pool configuration information may define a subset of available subframes and resource blocks for sidelink transmission from the sidelink anchor UE (e.g., 215a2). The receive resource pool configuration information may define a subset of available subframes and resource blocks for sidelink reception by the sidelink anchor UE. In some aspects, the RMSI includes transmission pattern information, such as S-SSB transmission patterns for rate matching purposes and/or RMSI transmission patterns for monitoring purposes.

In some aspects, the RMSI may include an active sidelink BWP configuration to assign an active sidelink BWP to a sidelink receiving UE. The sidelink anchor UE may dynamically assign a sidelink receiving UE to operate over a certain sidelink BWP (e.g., a certain portion of the system BW) using the active sidelink BWP configuration included in the RMSI. The sidelink receiving UE may monitor for signaling information from the sidelink anchor UE in an active sidelink BWP. The active sidelink BWP configuration may correspond to the initial sidelink BWP configuration in some embodiments, or the active sidelink BWP configuration may be different from the initial sidelink BWP configuration in other embodiments. In some aspects, the active sidelink BWP configuration and the initial sidelink BWP configuration may include separate numerologies. In this respect, the UE 215a2 may schedule the UE 215a1 for a sidelink communication in the active sidelink BWP.

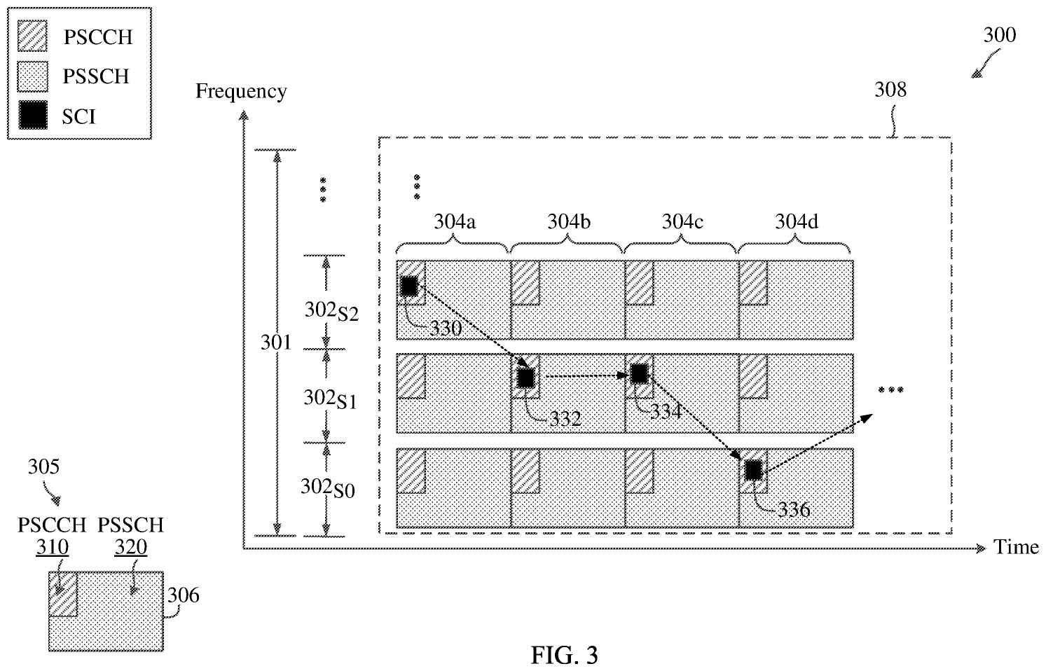

FIG. 3 illustrates a sidelink communication scheme 300 according to some aspects of the present disclosure. The scheme 300 may be employed by UEs such as the UEs 115 and/or 215 in a network such as the networks 100 and/or 200. In particular, sidelink UEs may employ the scheme 300 to communicate sidelink over a shared radio frequency band (e.g., in a shared spectrum or an unlicensed spectrum). The shared radio frequency band may be shared by multiple RATs as discussed in FIG. 2. In FIG. 3, the x-axis represents time in some arbitrary units, and the y-axis represents frequency in some arbitrary units.

In the scheme 300, a shared radio frequency band 301 is partitioned into a plurality of subchannels or frequency subbands 302 (shown as 302_{S0}, 302_{S1}, 302_{S2}, . . . ) in frequency and a plurality of sidelink frames 304 (shown as 304a, 304b, 304c, 304d, . . . ) in time for sidelink communication. The frequency band 301 may be at any suitable frequencies (e.g., at about 2.4 GHz, 5 GHz, or 6 GHz). The frequency band 301 may have any suitable BW and may be partitioned into any suitable number of frequency subbands 302. The number of frequency subbands 302 can be dependent on the sidelink communication BW requirement. The frequency band 301 may be at any suitable frequencies. In some aspects, the frequency band 301 is a 2.4 GHz unlicensed band and may have a bandwidth of about 80 megahertz (MHz) partitioned into about fifteen 5 MHz frequency subbands 302.

A sidelink UE (e.g., the UEs 115 and/or 215) may be equipped with a wideband receiver and a narrowband transmitter. For instance, the UE may utilize the narrowband transmitter to access a frequency subband 302_{S2} for sidelink transmission utilizing a frame structure 304. The frame structure 304 is repeated in each frequency subband 302. In some instances, there can be a frequency gap or guard band between adjacent frequency subbands 302 as shown in FIG. 3, for example, to mitigate adjacent band interference. Thus, multiple sidelink data may be communicated simultaneously in different frequency subbands 302 (e.g., FDM). The frame structure 304 is also repeated in time. For instance, the frequency subband 302_{S2} may be time-partitioned into a plurality of frames with the frame structure 304.

The frame structure 304 includes a sidelink resource 306 in each frequency subband 302. A legend 305 indicates the types of sidelink channels within a sidelink resource 306. The sidelink resource 306 may have a substantially similar structure as an NR sidelink resource. For instance, the sidelink resource 306 may include a number of subcarriers or RBs in frequency and a number of symbols in time. In some instances, the sidelink resource 306 may have a duration between about one millisecond (ms) to about 20 ms. Each sidelink resource 306 may include a PSCCH 310 and a PSSCH 320. The PSCCH 310 and the PSSCH 320 can be multiplexed in time and/or frequency. In the illustrated example of FIG. 3, for each sidelink resource 306, the PSCCH 310 is located during the beginning symbol(s) (e.g., about 1 symbol or about 2 symbols) of the sidelink resource 306 and occupies a portion of a corresponding frequency subband 302, and the PSSCH 320 occupies the remaining time-frequency resources in the sidelink resource 306. In some instances, a sidelink resource 306 may also include a physical sidelink feedback channel (PSFCH), for example, located during the ending symbol(s) of the sidelink resource 306. In general, a PSCCH 310, a PSSCH 320, and/or a PSFCH may be multiplexed in any suitable configuration within a sidelink resource 306.

As discussed above, the subject technology provides for a sidelink UE configured as a sidelink anchor UE (e.g., 115j, 215a2) for configuring resource allocations to other sidelink receiving UEs. As such, the sidelink anchor UE can configure the sidelink receiving UE with a resource pool configuration indicating resources in the frequency band 301 and/or the subbands 302 and/or timing information associated with the sidelink frames 304. For instance, the sidelink anchor UE can provide resource allocation information to sidelink receiving UEs. In some aspects, the sidelink anchor UE can communicate a transmit resource pool configuration in the RMSI, in which the transmit resource pool configuration indicates which radio resources are allocated to the sidelink anchor UE for the sidelink anchor UE to transmit a sidelink communication. In some aspects, the sidelink anchor UE can communicate a receive resource pool configuration in the RMSI, in which the receive resource pool configuration indicates which radio resources are allocated to the sidelink anchor UE for the sidelink anchor UE to receive a sidelink communication. In this respect, sidelink receiving UEs can receive and decode the physical communication channels (e.g., PSCCH 310, PSSCH 320) from the sidelink anchor UE based on the transmit resource pool configuration, and encode and transmit the PSCCH 310 and PSSCH 320 to the sidelink anchor UE based on the receive resource pool configuration.

In sidelink communication, in order for the sidelink receiving UEs to successfully decode the PSCCH 310 and PSSCH 320, information describing the specific resources assigned by the sidelink anchor UE for transmission and the transmission configuration can be carried in the sidelink control information, SCI. In this respect, control information for sidelink communication may be communicated in the form of SCI messages. The SCI message may be transmitted over the PSCCH 310, which carries the information related to the transmission of data over the PSSCH 320.

The SCI may inform the sidelink receiving UEs about a resource reservation interval, a frequency location of initial transmission and retransmission, a time gap between initial transmission and retransmission, and modulation and coding scheme (MCS) used to modulate the data transmitted over the PSSCH 320.

The SCI messages may be populated based on the modes of radio resource allocations (e.g., mode-1 RRA or mode-2 RRA). For mode-1 RRA, the SCI may be populated using higher layer information carried by L3 control signaling (e.g., RRC, and L1 control signaling configured at a cell, such as BS 215a). For mode-2 RRA, the SCI may be populated based on autonomous decisions taken by each sidelink anchor UE. The structure of the SCI message may include a frequency hopping flag field, a resource block assignment and hopping resource allocation field, a time resource pattern field, MCS field, a time advance field and a group destination identifier field. The structure of the SCI message may include other additional fields that are suitable to support V2X control signaling. The frequency hopping flag field and the resource block assignment and hopping resource allocation field may provide information for the sidelink receiving UEs to identify the RBs where the data channel (e.g., PSSCH 320) resides. The sidelink anchor UE may autonomously configure each of these two fields. The identified RBs may belong to a sidelink communication resource pool (e.g., PSSCH resource pool). The time resource pattern field may provide the time-domain resource allocation for the data channel (e.g., PSSCH 320), and in particular the potential subframes used for PSSCH transmission. The MCS field may provide the MCS used for the PSSCH 320, which may be autonomously selected by the sidelink anchor UE. The timing advance field may provide a sidelink time adjustment for mode-2 RRA or other applicable mode. The group destination identifier field may indicate a group of sidelink receiving UEs that are potentially interested in the transmitted message from the sidelink anchor UE. This may be used by the sidelink receiving UE to ignore messages destined to other groups of sidelink UEs.

In some aspects, the SCI messages may be processed with transport channel encoding to generate SCI message transport blocks, which are then followed with physical channel encoding to generate corresponding PSCCH blocks. The PSCCH blocks are carried on respective subframe resource units for transmission. The sidelink receiving UE may receive one or more resource units over respective subframes to recover the control signaling information, and can extract the data channel allocation and transmission configuration.

The PSCCH 310 can be used for carrying SCI 330. The PSSCH 320 can be used for carrying sidelink data. The sidelink data can be of various forms and types depending on the sidelink application. For instance, when the sidelink application is a V2X application, the sidelink data may carry V2X data (e.g., vehicle location information, traveling speed and/or direction, vehicle sensing measurements, etc.). Alternatively, when the sidelink application is an IIoT application, the sidelink data may carry IIoT data (e.g., sensor measurements, device measurements, temperature readings, etc.). The PSFCH can be used for carrying feedback information, for example, HARQ ACK/NACK for sidelink data received in an earlier sidelink resource 306.

In some aspects, the scheme 300 is used for synchronous sidelink communication. In other words, the sidelink UEs are synchronized in time and are aligned in terms of symbol boundary, sidelink resource boundary (e.g., the starting time of sidelink frames 304). The sidelink UEs may perform synchronization in a variety of forms, for example, based on sidelink SSBs received from a sidelink UE and/or NR-U SSBs received from a BS (e.g., the BSs 105 and/or 205) while in-coverage of the BS. In some aspects, the sidelink UE may be preconfigured with the resource pool 308 in the frequency band 301, for example, while in a coverage of a serving BS according to a mode-1 RRA configuration. The resource pool 308 may include a plurality of sidelink resources 306.

In an NR sidelink frame structure, the sidelink frames 304 in a resource pool 308 may be contiguous in time. A sidelink receiving UE (e.g., the UEs 115 and/or 215) may include, in SCI 330, a reservation for a sidelink resource 306 in a later sidelink frame 304. Thus, another sidelink UE (e.g., a UE in the same NR-U sidelink system) may perform SCI sensing in the resource pool 308 to determine whether a sidelink resource 306 is available or occupied. For instance, if the sidelink UE detected SCI indicating a reservation for a sidelink resource 306, the sidelink UE may refrain from transmitting in the reserved sidelink resource 306. If the sidelink UE determines that there is no reservation detected for a sidelink resource 306, the sidelink UE may transmit in the sidelink resource 306. As such, SCI sensing can assist a UE in identifying a target frequency subband 302 to reserve for sidelink communication and to avoid intra-system collision with another sidelink UE in the NR sidelink system. In some aspects, the UE may be configured with a sensing window for SCI sensing or monitoring to reduce intra-system collision.

In some aspects, the sidelink UE may be configured with a frequency hopping pattern. In this regard, the sidelink UE may hop from one frequency subband 302 in one sidelink frame 304 to another frequency subband 302 in another sidelink frame 304. In the illustrated example of FIG. 3, during the sidelink frame 304a, the sidelink UE transmits SCI 330 in the sidelink resource 306 located in the frequency subband 302_{S2} to reserve a sidelink resource 306 in a next sidelink frame 304b located at the frequency subband 302_{S1} Similarly, during the sidelink frame 304b, the sidelink UE transmits SCI 332 in the sidelink resource 306 located in the frequency subband 302_{S1} to reserve a sidelink resource 306 in a next sidelink frame 304c located at the frequency subband 302_{S1}. During the sidelink frame 304c, the sidelink UE transmits SCI 334 in the sidelink resource 306 located in the frequency subband 302_{S1} to reserve a sidelink resource 306 in a next sidelink frame 304d located at the frequency subband 302_{S0} During the sidelink frame 304d, the sidelink UE transmits SCI 336 in the sidelink resource 306 located in the frequency subband 302_{S0} The SCI 336 may reserve a sidelink resource 306 in a later sidelink frame 304.

The SCI can also indicate scheduling information and/or a destination identifier (ID) identifying a target sidelink receiving UE for the next sidelink resource 306. Thus, a sidelink UE may monitor SCI transmitted by other sidelink UEs. Upon detecting SCI in a sidelink resource 306, the sidelink UE may determine whether the sidelink UE is the target receiver based on the destination ID. If the sidelink UE is the target receiver, the sidelink UE may proceed to receive and decode the sidelink data indicated by the SCI. In some aspects, multiple sidelink UEs may simultaneously communicate sidelink data in a sidelink frame 304 in different frequency subband (e.g., via FDM). For instance, in the sidelink frame 304b, one pair of sidelink UEs may communicate sidelink data using a sidelink resource 306 in the frequency subband 302_{S2} while another pair of sidelink UEs may communicates sidelink data using a sidelink resource 306 in the frequency subband 302_{S1}.

The sidelink mechanisms described above with respect to FIG. 3 allow for a group of sidelink UEs to communicate in a shared frequency band (e.g., NR-U). In some aspects, these mechanisms may be used for standalone communications, including scenarios in which one or more of the UEs is not associated with a subscription. In some aspects, the mechanisms described in FIG. 3 may involve a dynamic sidelink discovery and communication process, which may involve significant coordination and/or standardization between the UEs. In some instances, it may be difficult or impractical to have such coordination between the sidelink UEs. For example, the UEs may be provided by different manufacturers and/or may be associated with different service providers. Accordingly, designating and indicating time/frequency resources for the PSSCH 320 and PSCCH 310 may be difficult. The present disclosure describes schemes and methods for indicating sidelink discovery information based on periodic synchronization communications, such as a sidelink synchronization signal block (S-SSB). For example, aspects of the present disclosure provide mechanisms for communicating a discovery signal, which may include RMSI as discussed above with respect to FIGS. 2 and 3, in an unoccupied portion of a subband carrying a synchronization communication, and time-aligned with the synchronization communication. Thus, a first sidelink UE (e.g., an anchor UE) may indicate discovery information to other sidelink UEs in a way that makes efficient use of resources associated with predefined synchronization signaling. In addition to improving network efficiency, these mechanisms may reduce or eliminate the concern for coordination between manufacturers and/or service providers to agree to standards for sidelink communication in shared frequency bands.

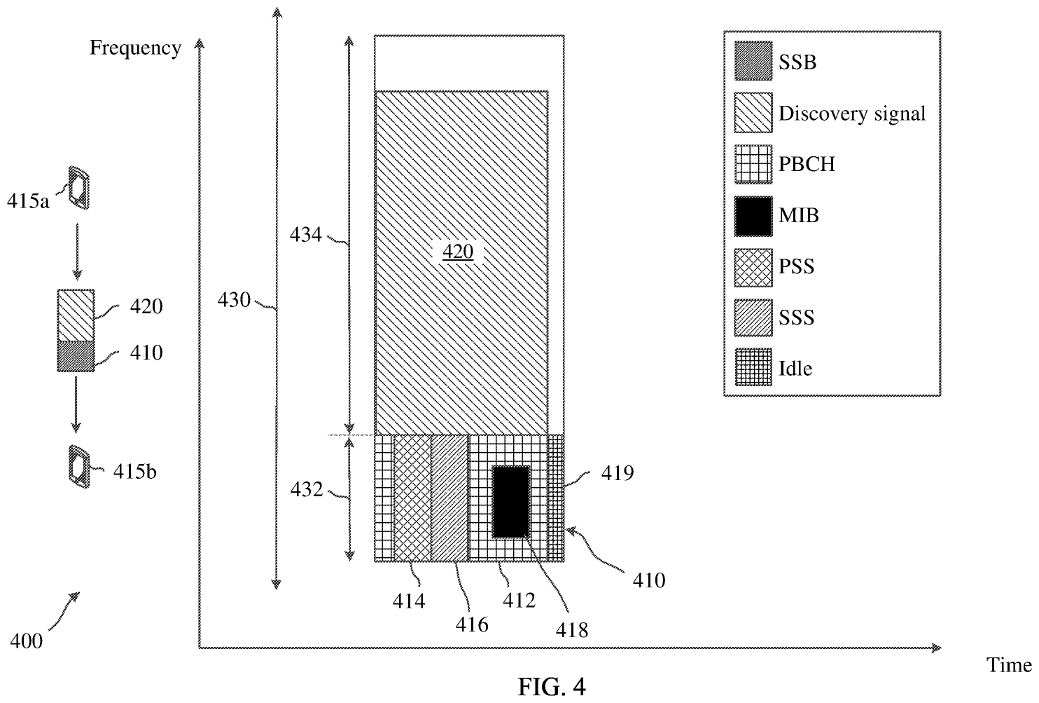

FIG. 4 illustrates a sidelink communication scheme 400 according to some aspects of the present disclosure. The scheme 300 may be employed by UEs such as the UEs 115 and/or 215 in a network such as the networks 100 and/or 200. In particular, sidelink UEs may employ the scheme 300 to communicate sidelink over a shared radio frequency band (e.g., in a shared spectrum or an unlicensed spectrum). The shared radio frequency band may be shared by multiple RATs as discussed in FIG. 2. In FIG. 4, the x-axis represents time in some arbitrary units, and the y-axis represents frequency in some arbitrary units.

In the scheme 400, a first sidelink UE 415a transmits, and a second sidelink UE 415b receives, a sidelink communication including a synchronization communication 410 and a discovery signal 420. The first sidelink UE 415a may transmit the sidelink communication in a shared frequency band (e.g., NR-U). The first sidelink UE 415a transmits the sidelink communication in a subband 430, which may be referred to as a channel. The subband 430 includes a first portion 432 and a second portion 434. The first portion 432 may include one or more first RBs, and the second portion 434 may include one or more second RBs. The one or more first RBs may be contiguous, and the one or more second RBs may be contiguous. Further, the one or more second RBs of the second portion 434 may be contiguous with the one or more first RBs of the first portion 432. In other aspects, there may be a gap of one or more RBs between the first portion 432 and the second portion 434. In one example, the subband 430 includes a total of 50 RBs, where the first portion 432 includes 11 RBs and the second portion 434 includes 30, 32, 36, 39, or any other suitable number of RBs. In another example, the subband 430 includes a total of 100 RBs, where the first portion 432 includes 11 RBs and the second portion 434 includes 60, 70, 72, 80, or any other suitable number of RBs. The RBs of the subband 430 may span a bandwidth, which may be 5 MHz, 10 MHz, 20 MHz, 40 MHz, or any other suitable bandwidth. In some aspects, the number of RBs in the subband may depend on a subcarrier spacing of the subband 430. The subcarrier spacing of the subband 430 may depend on the frequency range and/or carrier frequency of the subband 430. For example, for a first carrier frequency and bandwidth, the subcarrier frequency may be 15 kHz. For a second carrier frequency and bandwidth, the subcarrier frequency may be 30 kHz. For example, the subband 430 may include 50 RBs if the subcarrier spacing is 30 kHz, or 100 RBs if the subcarrier spacing is 15 kHz.

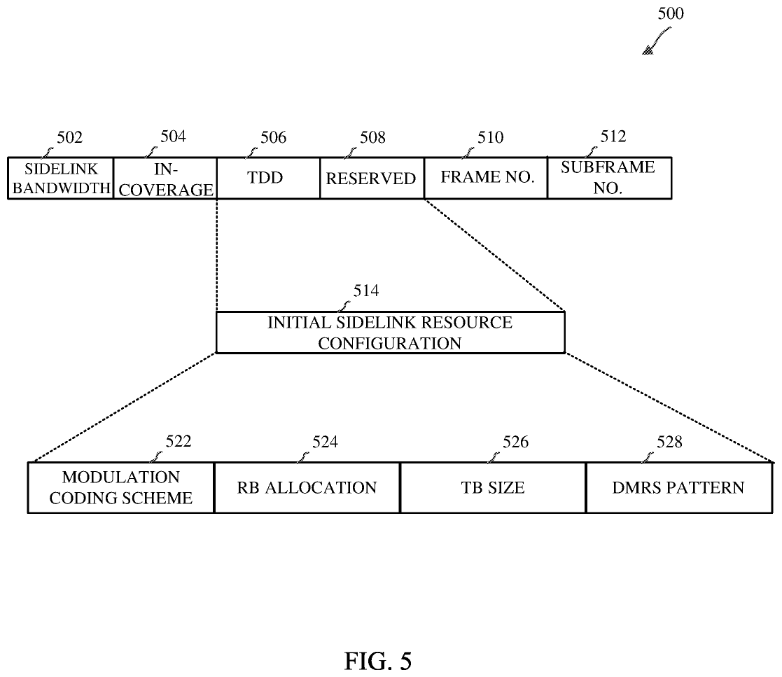

In the first portion 432 of the subband 430, the first sidelink UE 415a transmits, and the second sidelink UE 415b receives, a synchronization communication 410. The first sidelink UE 415a may transmit the synchronization communication 410 in the first 13 OFDM symbols of a slot, where the last OFDM symbol is an idle symbol or gap symbol 419. In an exemplary aspect, the synchronization communication 410 includes a synchronization signal block (SSB), such as a sidelink SSB (S-SSB). In this regard, the synchronization communication 410 includes a physical broadcast channel (PBCH) 412, a primary synchronization signal (PSS) 414, and a secondary synchronization signal (SSS) 416. The PBCH 412 carries control information 418. In some aspects, the PBCH 412 may be a physical sidelink broadcast channel (PSBCH). In an exemplary aspect, the control information 418 includes a master information block (MIB). The MIB may have the form of the MIB 500 described below with respect to FIG. 5, for example.

The control information or MIB 418 may be used to detect and/or decode the discovery signal 420. Accordingly, the discovery signal 420 may not carry its own control information, such as a physical sidelink control channel (PSCCH). Rather, the control information may be included or carried in the MIB 418. The control information 418 may include or indicate a modulation and coding scheme (MCS) of the discovery signal, a RB allocation of the discovery signal, a TB size of the discovery signal, a demodulation reference signal (DMRS) pattern of the discovery signal, and/or any other suitable control parameter associated with the discovery signal 420.

In the aspect of FIG. 4, the first sidelink UE 415a transmits the synchronization communication 410 in the first portion 432, where the first portion 432 is located at or near an edge of subband 430. In particular, the first portion 432 is at a lower or bottom edge of the subband 430 representative of the lower frequency subcarriers of the subband 430. In other aspects, the subband 430 may not be at an edge of the subband 430. For example, there may be one or more RBs on a first side of the first portion 432, and one or more RBs on the opposite second side of the first portion 434. In other aspects, the first portion 432 in which the first sidelink UE 415a transmits the synchronization communication 410 may be at or near a center frequency of the subband 430.

The first sidelink UE 415 may transmit, in the second portion 434 of the subband 430 and in the same slot used to transmit the synchronization communication 410, a discovery signal 420. The first sidelink UE 415a may transmit the discovery signal 420 such that the discovery signal 420 comprises a waveform similar or identical to a physical sidelink shared channel (PSSCH) communication. Accordingly, the first sidelink UE 415a may generate or prepare the discovery signal 420 to include a DMRS pattern, MCS, RB allocation, TB size, and/or any other waveform parameter based on the respective parameters of a PSSCH waveform. In some aspects, the information carried in the discovery signal 420 may be similar or identical to remaining minimum system information (RMSI). The discovery signal 420 may include or indicate sidelink connection configuration information including one or more sidelink communication parameters. For example, the discovery signal 420 may include or indicate at least one of a sidelink resource pool configuration, network service information associated with the first sidelink UE 415a, and/or UE identity information associated with the first sidelink UE 415a. Thus, the sidelink connection configuration may provide configuration information that can be used by a plurality of sidelink UEs to self-organize and coordinate transmissions, use of sidelink channel resources, detect and sync with other UEs, designate a coordinating or anchor UE, set up resource pools, and/or any other sidelink function.

In some aspects, transmitting the discovery signal 420 includes transmitting a physical shared channel. For example, the physical shared channel may be similar or identical to the PSSCH, in some aspects. In some aspects, the physical shared channel transmitted by the first sidelink UE may differ from a PSSCH in that control information (e.g., SCI) may be carried in the synchronization communication. For example, an MIB of a PBCH may carry the control information, as explained above. In some aspects, the first sidelink UE 415a may transmit multiple repetitions of the discovery signal 420 in a period of time. For example, the first sidelink UE 415a may transmit multiple repetitions of the discovery signal 420 within 80 ms intervals, or any other suitable interval. Accordingly, the second sidelink UE may perform soft decoding of the discovery signal 420 based on the multiple repetitions.

The second portion of the subband of the shared frequency band may include one or more RBs forming a different portion of the subband than the RBs used to transmit the synchronization communication. As explained above, the second portion of the subband may be contiguous with the first portion of the subband. For example, the first sidelink UE 415a may transmit the discovery signal 420 in a second plurality of RBs that is contiguous with a first plurality of RBs used to transmit the synchronization communication. In some aspects, the number of RBs used by the first sidelink UE to transmit the discovery signal 420 may depend on the subcarrier spacing of the shared frequency band. For example, if the shared frequency band is associated with a subcarrier spacing of 30 kHz, the first sidelink UE 415a may transmit the discovery signal 420 in a contiguous group of 36 RBs, and may transmit the synchronization communication in a contiguous group of 11 RBs. In another example, if the shared frequency band is associated with a subcarrier spacing of 15 kHz, the first sidelink UE may transmit the discovery signal 420 in a contiguous group of 72 RBs. However, it will be understood that these values are examples, and that the first sidelink UE 415a may transmit the discovery sidelink in any suitable number of RBs.

Further, as mentioned above, the first sidelink UE 415a transmits the discovery signal 420 in the first slot, which is the same slot used to transmit the synchronization communication. In some aspects, the first sidelink UE 415b may transmit, and the second sidelink UE 415b may receive, the discovery signal 420 in the same 13 OFDM symbols used to transmit the synchronization communication, where the 14^{th} symbol is an idle symbol or gap symbol. Accordingly, the discovery signal 420 and the synchronization communication may be aligned in the time domain.

In some aspects, the first sidelink UE 415a transmits the discovery signal 420 such that it is quasi co-located (QCL) with the synchronization communication. Further, in some aspects, the first sidelink UE 415a may transmit the discovery signal 420 using an antenna port that is the same antenna portion used to transmit the synchronization communication. In some aspects, by transmitting the discovery signal 420 such that it is aligned in time and QCL with the synchronization communication, and such that the discovery signal 420 occupies a portion of the subband that is not occupied by the synchronization communication (e.g., S-SSB), the first sidelink UE 415a may use network resources efficiently to send sidelink configuration information for sidelink coordination. Further, transmitting the discovery signal 420 in this way may provide a flexible and inclusive discovery signal 420ing scheme that can be used by devices associated with a variety of network service providers and equipment manufacturers. For example, the scheme 400 may be used by UEs that have no network subscription, in some aspects.

FIG. 5 is a simplified block diagram of an exemplary frame structure of a sidelink master information block (MIB) 500 according to some aspects of the present disclosure. The frame structure includes a sidelink bandwidth field 502, in-coverage indicator field 504, time-division duplex (TDD) configuration field 506, reserved field 508, frame number field 510, and subframe number field 512. Not all of the depicted frame structure fields may be included, however, and one or more implementations may include additional frame structure fields not shown in the figure. Variations in the arrangement and type of the frame structure fields may be made without departing from the scope of the claims as set forth herein. Additional frame structure fields, different frame structure fields, or fewer frame structure fields may be provided.

As described herein, the sidelink master information block can carry the system parameter information. The sidelink master information block may be analogous to MIB in NR and NR-U systems. In NR or NR-U systems, the MIB may contain an 8-bit information field that configures CORESET 0 and Type-0 PDCCH monitoring. However, in sidelink communication, the sidelink master information block may not contain such bit field information that corresponds to the CORESET 0 and Type-0 PDCCH monitoring. In some embodiments, the SL-MIB in the S-SSB may repurpose the 8-bit information field to indicate the location of a discovery signal. For example, multiple bits in the reserved field 508 and/or the TDD configuration field 506 of the sidelink master information block can be repurposed by the sidelink UE into an initial sidelink resource configuration field 514.

The sidelink master information block may be mapped to a reference subframe at a specific frequency and/or time resource allocation. As depicted in FIG. 5, the frame structure of the sidelink master information block 510 may include a 40-bit sequence. In some instances, the sidelink bandwidth field 502 may provide the bandwidth mode (e.g., 5, 10, 15, 20 MHz). The in-coverage indictor field 504 may inform a sidelink receiving UE about the coverage status of the sidelink UE (e.g., in-coverage, partial coverage, out-of-coverage). The frame field 510 and subframe field 512 may provide timing reference information in the frame and subframe time-scales, respectively.

As depicted in FIG. 5, the TDD configuration field 506 and the reserved field 508 have been repurposed into the initial sidelink configuration field 514. However, in other aspects, the initial sidelink resource configuration field 514 may be a separate field from the TDD configuration field 506 and/or the reserved field 508. In some instances, the initial sidelink configuration field 514 may include a pointer that indicates a location to the discovery signal for use by the sidelink receiving UE to recover the discovery signal after locating the S-SSB. In this regard, the initial sidelink configuration field 514 includes an RB allocation field 524 indicating at least one of a starting RB of the discovery signal, or a range of RBs associated with the discovery signal.

The location or RB allocation of the discovery signal may be indicated in the field 524 with respect to the S-SSB, in some aspects. In some aspects, the sidelink UE may allocate a bit field in the initial sidelink resource configuration field 514 to indicate at least one of a plurality of predefined sets of initial sidelink BWP configurations. The initial sidelink configuration field 514 includes a modulation coding scheme (MCS) field 522, a transport block (TB) size field 526, and a demodulation reference signal (DMRS) pattern field 528. The modulation coding scheme field 522 may indicate the type of modulation used to modulate the discovery signal (e.g., QPSK). The TB size field 526 may indicate the number of bits of each TB carried in the discovery signal. The DMRS pattern field 528 may indicate the DMRS pattern.

In some aspects, one or more of the fields 522, 524, 526, and 528 may include one bit, or a few bits, indicating a selection of one option for MCS, RB allocation, TB size, DMRS pattern, etc. For example, the sidelink UEs may be configured with two or more MCSs, RB allocations, TB sizes, and/or DMRS patterns, and the values of the fields 522, 524, 526, 528 may indicate which of the options is selected for the discovery signal. In other aspects, one or more of the fields 522, 524, 526, 528 may include or carry an absolute value, such as a numerical value, to indicate the respective parameter. For example, the value of the TB size field 526 may comprise a plurality of bits whose value indicates the TB size associated with the discovery signal. In another aspect, the value of the RB allocation field 524 may indicate the value of the starting and/or ending RB of the discovery signal, with respect to the RBs of the S-SSB.

After decoding the sidelink master information block, the sidelink receiving UE may receive and recover the discovery signal based on the pointer provided within the initial sidelink configuration field 514. In other instances, the UE may allocate a bit location in the initial sidelink configuration field 514 to include an indication of whether the sidelink master information block includes the discovery signal. For example, the sidelink master information block indication may indicate that there is no discovery signal and the sidelink receiving UE may not attempt to monitor for the discovery signal.

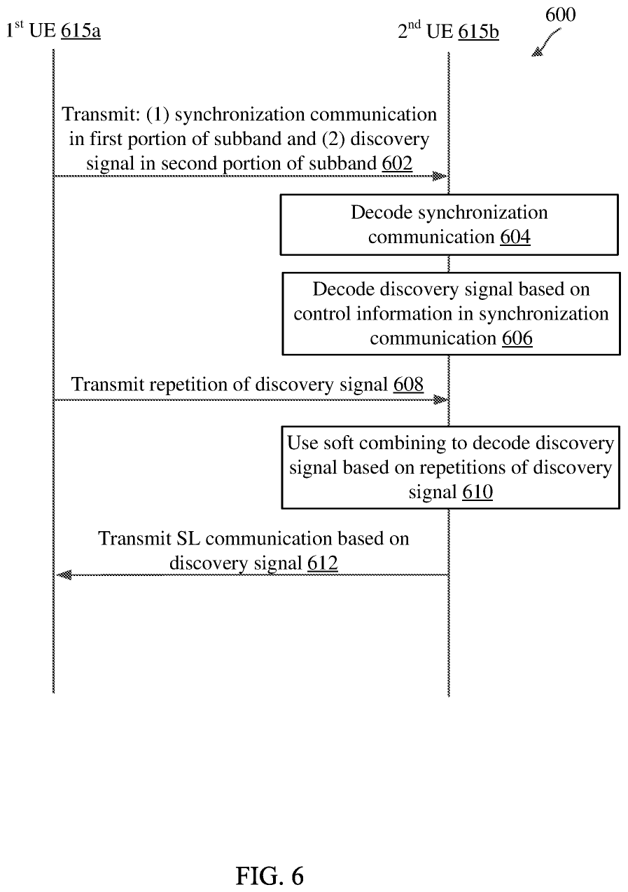

FIG. 6 is a signaling diagram of a method 600 for sidelink communication with discovery signaling, according to some aspects of the present disclosure. The method 600 is performed by a first UE 615a and a second UE 615b. Each of the first UE 615a and second UE 615b may be one of the UEs 115 in the network 100, one of the UEs 215 shown in FIG. 2, and/or one of the UEs 415 shown in FIG. 4. In particular, sidelink UEs may employ the method 600 to communicate sidelink over a shared radio frequency band (e.g., in a shared spectrum or an unlicensed spectrum). The shared radio frequency band may be shared by multiple RATs as discussed in FIG. 2. In some aspects, one of the UEs, such as the first UE 615a, may serve as an anchor UE for coordinating sidelink communications between one or more non-anchor UEs (e.g., the second UE 615b).

At action 602, the first UE 615a transmits, and the second UE 615b receives, a synchronization communication in a first portion of a subband, and a discovery signal in a second portion of the subband. The first UE 615a may transmit the synchronization communication and the discovery signal in a same slot, such that the synchronization communication and the discovery signal are aligned in time. The synchronization communication may include or indicate control information that can be used by the second UE 615b to decode the discovery signal. In some aspects, action 602 includes transmitting a synchronization signal block (SSB), which includes a physical broadcast channel (PBCH), a primary synchronization signal (PSS), and a secondary synchronization signal (SSS). The SSB may be a sidelink SSB (S-SSB), for example. In some aspects, the first UE 615a may transmit a PBCH carrying a master information block (MIB). The MIB may have a structure similar or identical to the structure of the MIB 500 shown in FIG. 5. For example, the MIB may include a sidelink resource configuration that includes or indicates a modulation coding scheme, a resource block (RB) allocation, a transport block (TB) size, a demodulation reference signal (DMRS) pattern, and/or any other suitable parameter associated with the discovery signal. In some aspects, the first UE 615a transmits, in the PBCH, an indication of a first RB of the discovery signal and a size (e.g., in RBs) of the discovery signal. In some aspects, the first UE 615a may also indicate the location and/or periodicity of reference signals carried in the discovery signal. The synchronization information may include any suitable parameter or information to assist the second UE 615b in detecting and decoding the discovery signal.

The first portion of the subband may include one or more contiguous RBs in the subband. In some aspects, the subband may be described as a channel having a bandwidth of 20 MHz, 80 MHz, 10 MHz, or any other suitable bandwidth, both greater or smaller. The bandwidth of the subband may also be described or defined in terms of RBs. For example, the subband may include 50 RBs, 100 RBs, or any other suitable number of RBs, both greater or smaller. The first UE 615a may transmit the synchronization communication such that the synchronization communication occupies the first portion of the plurality of RBs in the subband. In one aspect, the first portion of the subband includes 11 RBs. In some aspects, at least one of the RBs may be at or near an edge (e.g., lowest frequency or highest frequency) of the subband. For example, a first RB of the first portion of the subband may be a first RB of the subband. In other aspects, the first RB of the first portion may not the be first RB of the subband. For example, the subband may include one or more RBs on either side of the first portion of the subband, where the synchronization communication is transmitted in the first portion of the subband.

The first UE 615a may transmit the discovery signal such that the discovery signal comprises a waveform similar or identical to a PSSCH communication. Accordingly, the first UE 615a may generate or prepare the discovery signal to include a DMRS pattern, MCS, RB allocation, TB size, and/or any other waveform parameter based on the respective parameters of a PSSCH waveform. In some aspects, the information carried in the discovery signal may be similar or identical to remaining minimum system information (RMSI). The discovery signal may include or indicate sidelink connection configuration information including one or more sidelink communication parameters. For example, the discovery signal may include or indicate at least one of a sidelink resource pool configuration, network service information associated with the first UE 615a, and/or UE identity information associated with the first UE 615a. Thus, the sidelink connection configuration may provide configuration information that can be used by a plurality of sidelink UEs to self-organize and coordinate transmissions, use of sidelink channel resources, detect and sync with other UEs, designate a coordinating or anchor UE, set up resource pools, and/or any other sidelink function.

In some aspects, transmitting the discovery signal includes transmitting a physical shared channel. For example, the physical shared channel may be similar or identical to the physical sidelink shared channel (PSSCH), in some aspects. In some aspects, the physical shared channel transmitted by the first UE 615a may differ from a PSSCH in that control information (e.g., SCI) may be carried in the synchronization communication. For example, the MIB of the PBCH may carry the control information, as explained above.

The second portion of the subband of the shared frequency band may include one or more RBs forming a different portion of the subband than the RBs used to transmit the synchronization communication. As explained above, the second portion of the subband may be contiguous with the first portion of the subband. For example, the first UE 615a may transmit the discovery signal in a second plurality of RBs that is contiguous with a first plurality of RBs used to transmit the synchronization communication. In some aspects, the number of RBs used by the first UE 615a to transmit the discovery signal may depend on the subcarrier spacing of the shared frequency band. For example, if the shared frequency band is associated with a subcarrier spacing of 30 kHz, the first UE 615a may transmit the discovery signal in a contiguous group of 36 RBs, and may transmit the synchronization communication in a contiguous group of 11 RBs. In another example, if the shared frequency band is associated with a subcarrier spacing of 15 kHz, the first UE 615a may transmit the discovery signal in a contiguous group of 72 RBs. However, it will be understood that these values are examples, and that the first UE 615a may transmit the discovery sidelink in any suitable number of RBs.

Further, as mentioned above, the first UE 615a transmits the discovery signal in the first slot, which is the same slot used to transmit the synchronization communication. In some aspects, the first UE 615a may transmit, and the second UE 615b may receive, the discovery signal in the same 13 OFDM symbols used to transmit the synchronization communication, where the 14^{th} symbol is an idle symbol, or gap symbol. Accordingly, the discovery signal and the synchronization communication may be aligned in the time domain.

In some aspects, the first UE 615a transmits the discovery signal such that it is quasi co-located (QCL) with the synchronization communication. Further, in some aspects, the first UE 615a may transmit the discovery signal using an antenna port that is the same antenna portion used to transmit the synchronization communication.

At action 604, the second UE 615b decodes the synchronization communication. In some aspects, decoding the synchronization communication includes decoding a S-SSB. The S-SSB may include a PBCH (e.g., a PSBCH), a PSS, and a SSS. The PBCH may include the control information as discussed above. Accordingly, action 604 may include decoding or detecting an MIB for example that includes control information used by the second UE 615b to decode the discovery signal.

At action 606, the second UE 615b decodes the discovery signal based on the control information in the synchronization communication. For example, the second UE 615b may decode or detect the discovery signal based on a sidelink resource configuration carried in the MIB. The sidelink resource configuration may include or indicate a modulation coding scheme, a resource block (RB) allocation, a transport block (TB) size, a demodulation reference signal (DMRS) pattern, and/or any other suitable parameter associated with the discovery signal. The second UE 615b may decode or detect the discovery signal based on one or more of these parameters.

At action 608, the first UE 615a transmits, and the second UE 615b receives, a repetition of the discovery signal. In this regard, the first UE 615a may transmit multiple repetitions of the discovery signal in a period of time. For example, the first UE 615a may transmit multiple repetitions of the discovery signal within 80 ms intervals, or any other suitable interval.

At action 610, the second UE 615b uses soft decoding of the discovery signal based on the multiple repetitions of the discovery signal.

At action 612, the second UE 615b transmits, and the first UE 615a receives, a sidelink communication based on the discovery signal. For example, as discussed above, the discovery signal may indicate sidelink connection configuration information including one or more sidelink communication parameters. For example, the discovery signal may include or indicate at least one of a sidelink resource pool configuration, network service information associated with the first UE 615a, and/or UE identity information associated with the first UE 615a. Thus, the sidelink connection configuration may provide configuration information that can be used by a plurality of sidelink UEs to self-organize and coordinate transmissions, use of sidelink channel resources, detect and sync with other UEs, designate a coordinating or anchor UE, set up resource pools, and/or any other sidelink function. The second UE 615b may use this information to determine whether to initiate sidelink communications with the first UE 615a, and/or to determine what time/frequency resources to use for a sidelink communication.

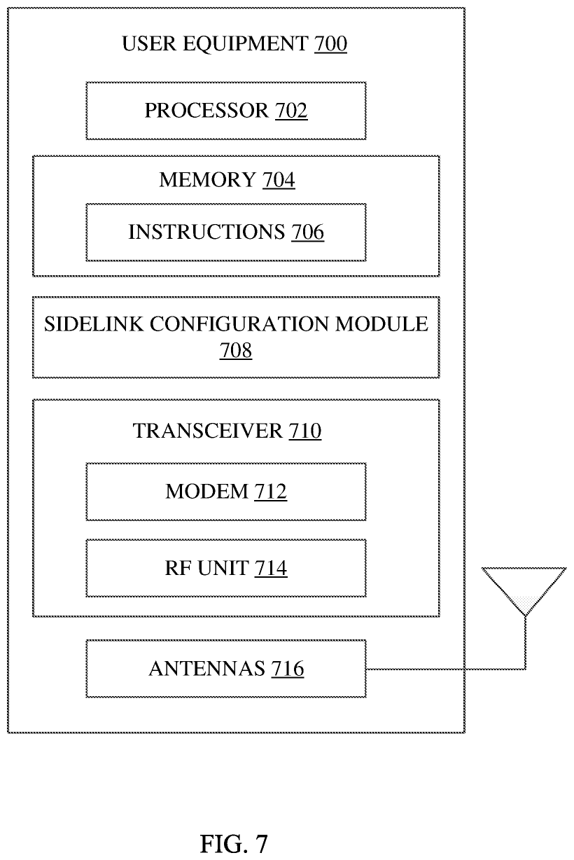

FIG. 7 is a block diagram of an exemplary sidelink UE 700 according to some aspects of the present disclosure. The sidelink UE 700 may be a UE 115 in the network 100 as discussed above in FIG. 1, a UE 215 discussed above in FIG. 2, or a UE 415 as discussed above in FIG. 4. As shown, the sidelink UE 700 may include a processor 702, a memory 704, a sidelink configuration module 708, a transceiver 710 including a modem subsystem 712 and a RF unit 714, and one or more antennas 716. These elements may be in direct or indirect communication with each other, for example via one or more buses.

The processor 702 may have various features as a specific-type processor. For example, these may include a CPU, a DSP, an ASIC, a controller, a FPGA device, another hardware device, a firmware device, or any combination thereof configured to perform the operations described herein. The processor 702 may also be implemented as a combination of computing devices, e.g., a combination of a DSP and a microprocessor, a plurality of microprocessors, one or more microprocessors in conjunction with a DSP core, or any other such configuration.

The memory 704 may include a cache memory (e.g., a cache memory of the processor 702), RAM, MRAM, ROM, PROM, EPROM, EEPROM, flash memory, a solid state memory device, one or more hard disk drives, memristor-based arrays, other forms of volatile and non-volatile memory, or a combination of different types of memory. In some aspects, the memory 704 may include a non-transitory computer-readable medium. The memory 704 may store instructions 706. The instructions 706 may include instructions that, when executed by the processor 702, cause the processor 702 to perform operations described herein, for example, aspects of FIGS. 1-6, 8 and/or 9. Instructions 706 may also be referred to as code, which may be interpreted broadly to include any type of computer-readable statement(s).

The sidelink configuration module 708 may be implemented via hardware, software, or combinations thereof. For example, the sidelink configuration module 708 may be implemented as a processor, circuit, and/or instructions 706 stored in the memory 704 and executed by the processor 702. In some instances, the sidelink configuration module 708 can be integrated within the modem subsystem 712. For example, the sidelink configuration module 708 can be implemented by a combination of software components (e.g., executed by a DSP or a general processor) and hardware components (e.g., logic gates and circuitry) within the modem subsystem 712.

The sidelink configuration module 708 may be used for various aspects of the present disclosure, for example, aspects of FIGS. 1-6, 8 and/or 9. For instance, the sidelink configuration module 708 is configured to transmit, to a second sidelink UE in a first slot and a first portion of a subband of a shared frequency band, a synchronization communication. The synchronization communication may indicate a location of a discovery signal in a second portion of the subband of the shared frequency band. In some aspects, transmitting the synchronization communication includes transmitting a synchronization signal block (SSB), which includes a physical broadcast channel (PBCH), a primary synchronization signal (PSS), and a secondary synchronization signal (SSS). The SSB may be a sidelink SSB (S-SSB), for example. In some aspects, the sidelink configuration module 708 may be configured to transmit a PBCH carrying a master information block (MIB). The MIB may have a structure similar or identical to the structure of the MIB 500 shown in FIG. 5. For example, the MIB may include a sidelink resource configuration that includes or indicates a modulation coding scheme, a resource block (RB) allocation, a transport block (TB) size, a demodulation reference signal (DMRS) pattern, and/or any other suitable parameter associated with the discovery signal. In some aspects, the sidelink configuration module 708 may be configured to transmit, in the PBCH, an indication of a first RB of the discovery signal and a size (e.g., in RBs) of the discovery signal. In some aspects, the sidelink configuration module 708 may be configured to indicate the location and/or periodicity of reference signals carried in the discovery signal. The synchronization information may include any suitable parameter or information to assist the second sidelink UE in detecting and decoding the discovery signal.

The first portion of the subband may include one or more contiguous RBs in the subband. In some aspects, the subband may be described as a channel having a bandwidth of 20 MHz, 80 MHz, 10 MHz, or any other suitable bandwidth, both greater or smaller. The bandwidth of the subband may also be described or defined in terms of RBs. For example, the subband may include 50 RBs, 100 RBs, or any other suitable number of RBs, both greater or smaller. The sidelink configuration module 708 may be configured to transmit the synchronization communication such that the synchronization communication occupies the first portion of the plurality of RBs in the subband. In one aspect, the first portion of the subband includes 11 RBs. In some aspects, at least one of the RBs may be at or near an edge (e.g., lowest frequency or highest frequency) of the subband. For example, a first RB of the first portion of the subband may be a first RB of the subband. In other aspects, the first RB of the first portion may not the be first RB of the subband. For example, the subband may include one or more RBs on either side of the first portion of the subband, where the synchronization communication is transmitted in the first portion of the subband.

The sidelink configuration module 708 may be further configured to transmit, to the second sidelink UE in the first slot and in the second portion of the subband of the shared frequency band, the discovery signal. The sidelink configuration module 708 may be configured to transmit the discovery signal such that the discovery signal comprises a waveform similar or identical to a PSSCH communication. Accordingly, the sidelink configuration module 708 may be configured to generate or prepare the discovery signal to include a DMRS pattern, MCS, RB allocation, TB size, and/or any other waveform parameter based on the respective parameters of a PSSCH waveform. In some aspects, the information carried in the discovery signal may be similar or identical to remaining minimum system information (RMSI). The discovery signal may include or indicate sidelink connection configuration information including one or more sidelink communication parameters. For example, the discovery signal may include or indicate at least one of a sidelink resource pool configuration, network service information associated with the sidelink UE 700, and/or UE identity information associated with the sidelink UE 700. Thus, the sidelink connection configuration may provide configuration information that can be used by a plurality of sidelink UEs to self-organize and coordinate transmissions, use of sidelink channel resources, detect and sync with other UEs, designate a coordinating or anchor UE, set up resource pools, and/or any other sidelink function.

In some aspects, transmitting the discovery signal includes transmitting a physical shared channel. For example, the physical shared channel may be similar or identical to the physical sidelink shared channel (PSSCH), in some aspects. In some aspects, the physical shared channel may differ from a PSSCH in that control information (e.g., SCI) may be carried in the synchronization communication. For example, the MIB of the PBCH may carry the control information, as explained above. In some aspects, the sidelink configuration module 708 may be configured to transmit multiple repetitions of the discovery signal in a period of time. For example, the sidelink configuration module 708 may be configured to transmit multiple repetitions of the discovery signal within 80 ms intervals, or any other suitable interval. Accordingly, the second sidelink UE may perform soft decoding of the discovery signal based on the multiple repetitions.

The second portion of the subband of the shared frequency band may include one or more RBs forming a different portion of the subband than the RBs used to transmit the synchronization communication. As explained above, the second portion of the subband may be contiguous with the first portion of the subband. For example, the sidelink configuration module 708 may be configured to transmit the discovery signal in a second plurality of RBs that is contiguous with a first plurality of RBs used to transmit the synchronization communication. In some aspects, the number of RBs used by the sidelink configuration module 708 to transmit the discovery signal may depend on the subcarrier spacing of the shared frequency band. For example, if the shared frequency band is associated with a subcarrier spacing of 30 kHz, the sidelink configuration module 708 may be configured to transmit the discovery signal in a contiguous group of 36 RBs, and may transmit the synchronization communication in a contiguous group of 11 RBs. In another example, if the shared frequency band is associated with a subcarrier spacing of 15 kHz, the sidelink configuration module 708 may be configured to transmit the discovery signal in a contiguous group of 72 RBs. However, it will be understood that these values are examples, and that the sidelink configuration module 708 may be configured to transmit the discovery sidelink in any suitable number of RBs.

Further, as mentioned above, the sidelink configuration module 708 may be configured to transmit the discovery signal in the first slot, which is the same slot used to transmit the synchronization communication. In some aspects, the sidelink configuration module 708 may be configured to transmit, and the second sidelink UE may receive, the discovery signal in the same 13 OFDM symbols used to transmit the synchronization communication, where the 14^{th} symbol is an idle symbol, or gap symbol. Accordingly, the discovery signal and the synchronization communication may be aligned in the time domain.

In some aspects, the sidelink configuration module 708 may be configured to transmit the discovery signal such that it is quasi co-located (QCL) with the synchronization communication. Further, in some aspects, the sidelink configuration module 708 may be configured to transmit the discovery signal using an antenna port that is the same antenna portion used to transmit the synchronization communication.

In another aspect, the sidelink configuration module 708 may be used to receive and/or detect a discovery signal. For example, the sidelink configuration module 708 may be configured to receive, from a second sidelink UE in a first slot and a first portion of a subband of a shared frequency band, a synchronization communication. The synchronization communication may indicate a location of a discovery signal in a second portion of the subband of the shared frequency band. In some aspects, receiving the synchronization communication includes receiving a synchronization signal block (SSB), which includes a physical broadcast channel (PBCH), a primary synchronization signal (PSS), and a secondary synchronization signal (SSS). The SSB may be a sidelink SSB (S-SSB), for example. In some aspects, the sidelink configuration module 708 may be configured to receive a PBCH carrying a master information block (MIB). The MIB may have a structure similar or identical to the structure of the MIB 500 shown in FIG. 5. For example, the MIB may include a sidelink resource configuration that includes or indicates a modulation coding scheme, a resource block (RB) allocation, a transport block (TB) size, a demodulation reference signal (DMRS) pattern, and/or any other suitable parameter associated with the discovery signal. In some aspects, the sidelink configuration module 708 may be configured to receive a PBCH including an indication of a first RB of the discovery signal and a size (e.g., in RBs) of the discovery signal. In some aspects, the sidelink configuration module 708 may be configured to receive an indication of the location and/or periodicity of reference signals carried in the discovery signal. The synchronization information may include any suitable parameter or information to assist the first UE in detecting and decoding the discovery signal.

In some aspects, the sidelink configuration module 708 may be further configured to receive, from the second sidelink UE in the first slot and in the second portion of the subband of the shared frequency band, the discovery signal. The sidelink configuration module 708 may be configured to receive the discovery signal based on a waveform similar or identical to a PSSCH communication. Accordingly, the sidelink configuration module 708 may be configured to detect or decode the discovery signal based on a DMRS pattern, MCS, RB allocation, TB size, and/or any other waveform parameter based on the respective parameters of a PSSCH waveform. In some aspects, the information carried in the discovery signal may be similar or identical to remaining minimum system information (RMSI). The discovery signal may include or indicate one or more sidelink communication parameters. For example, the discovery signal may provide configuration information that can be used by a plurality of sidelink UEs to self-organize and coordinate transmissions, use of sidelink channel resources, detect and sync with other UEs, provide a coordinating or anchor UE, set up resource pools, and/or any other sidelink function.

In some aspects, receiving the discovery signal includes transmitting a physical shared channel. For example, the physical shared channel may be similar or identical to the physical sidelink shared channel (PSSCH), in some aspects. The physical shared channel may differ from a PSSCH in that control information (e.g., SCI) may be carried in the synchronization communication. For example, the MIB of the PBCH may carry the control information, as explained above. In some aspects, the sidelink configuration module 708 may be configured to receive multiple repetitions of the discovery signal in a period of time. For example, the sidelink configuration module 708 may be configured to receive multiple repetitions of the discovery signal within 80 ms intervals, or any other suitable interval. Accordingly, the sidelink configuration module 708 may be configured to perform soft decoding of the discovery signal based on the multiple repetitions.

As shown, the transceiver 710 may include the modem subsystem 712 and the RF unit 714. The transceiver 710 can be configured to communicate bi-directionally with other devices, such as the UEs 115 and/or 700 and/or another core network element. The modem subsystem 712 may be configured to modulate and/or encode data according to a MCS, e.g., a LDPC coding scheme, a turbo coding scheme, a convolutional coding scheme, a polar coding scheme, a digital beamforming scheme, etc. The RF unit 714 may be configured to process (e.g., perform analog to digital conversion or digital to analog conversion, etc.) modulated/encoded data (e.g., PDCCH, PDSCH, SSBs, sidelink configuration, sidelink resource pool configuration, SSBs, frequency hopping patterns for sidelink communication) from the modem subsystem 712 (on outbound transmissions) or of transmissions originating from another source such as a UE 115 and/or UE 700. The RF unit 714 may be further configured to perform analog beamforming in conjunction with the digital beamforming. Although shown as integrated together in transceiver 710, the modem subsystem 712 and/or the RF unit 714 may be separate devices that are coupled together at the UE 115 to enable the UE 115 to communicate with other devices.

The RF unit 714 may provide the modulated and/or processed data, e.g. data packets (or, more generally, data messages that may contain one or more data packets and other information), to the antennas 716 for transmission to one or more other devices. The RF unit 714 may process the modulated and/or processed data and generate corresponding time-domain waveforms using SC-FDMA modulation prior to transmission via the antennas 716. The antennas 716 may further receive data messages transmitted from other devices and provide the received data messages for processing and/or demodulation at the transceiver 710. The transceiver 710 may provide the demodulated and decoded data to the sidelink configuration module 708 for processing. The antennas 716 may include multiple antennas of similar or different designs to sustain multiple transmission links.

In an aspect, the sidelink UE 700 can include multiple transceivers 710 implementing different RATs (e.g., NR and LTE). In an aspect, the sidelink UE 700 can include a single transceiver 710 implementing multiple RATs (e.g., NR and LTE). In an aspect, the transceiver 710 can include various components, where different combinations of components can implement different RATs.

In some embodiments, the sidelink UE 700 can provide the discovery signal in the form of the RMSI. In some aspects, the RMSI includes additional system parameter information that is different from, at least a portion of, the system parameter information in the SL-MIB. In some aspects, the transceiver 710 can communicate the RMSI in one or more PSCCHs of the plurality of PSCCHs, and the transceiver 710 can further communicate the sidelink data in at least one PSSCH of the plurality of PSSCHs.

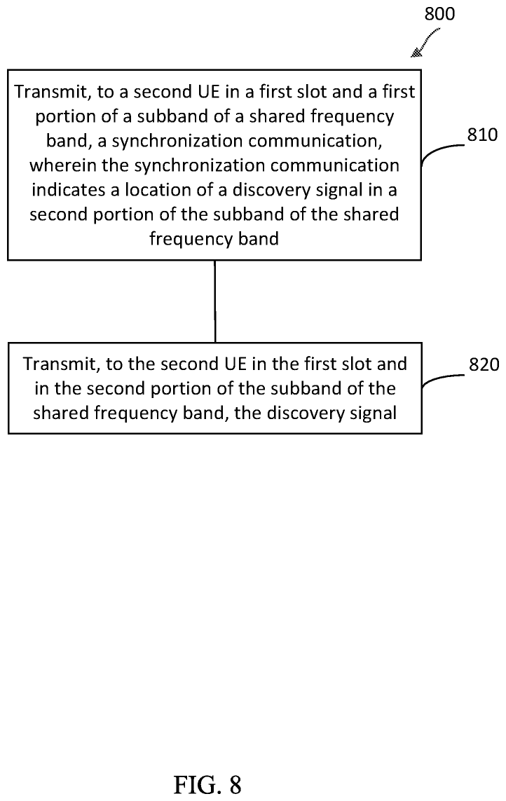

FIG. 8 is a flow diagram of an anchor node discovery process according to some aspects of the present disclosure. Aspects of the process 800 can be executed by a computing device (e.g., a processor, processing circuit, and/or other suitable component) of a wireless communication device or other suitable means for performing the steps. For example, a wireless communication device, such as the UEs 115, 215, 415, and/or 700, may utilize one or more components, such as the processor 702, the memory 704, the sidelink configuration module 708, the transceiver 710, the modem 712, and the one or more antennas 716, to execute the steps of process 800. As illustrated, the process 800 includes a number of enumerated steps, but aspects of the process 800 may include additional steps before, after, and in between the enumerated steps. In some aspects, one or more of the enumerated steps may be omitted or performed in a different order.

At block 810, a first sidelink UE (e.g., UE 115j) transmits, to a second sidelink UE in a first slot and a first portion of a subband of a shared frequency band, a synchronization communication. The synchronization communication may indicate a location of a discovery signal in a second portion of the subband of the shared frequency band. In some aspects, transmitting the synchronization communication includes transmitting a synchronization signal block (SSB), which includes a physical broadcast channel (PBCH), a primary synchronization signal (PSS), and a secondary synchronization signal (SSS). The SSB may be a sidelink SSB (S-SSB), for example. In some aspects, the first sidelink UE may transmit a PBCH carrying a master information block (MIB). The MIB may have a structure similar or identical to the structure of the MIB 500 shown in FIG. 5. For example, the MIB may include a sidelink resource configuration that includes or indicates a modulation coding scheme, a resource block (RB) allocation, a transport block (TB) size, a demodulation reference signal (DMRS) pattern, and/or any other suitable parameter associated with the discovery signal. In some aspects, the first sidelink UE transmits, in the PBCH, an indication of a first RB of the discovery signal and a size (e.g., in RBs) of the discovery signal. In some aspects, the first sidelink UE may also indicate the location and/or periodicity of reference signals carried in the discovery signal. The synchronization information may include any suitable parameter or information to assist the second sidelink UE in detecting and decoding the discovery signal.

The first portion of the subband may include one or more contiguous RBs in the subband. In some aspects, the subband may be described as a channel having a bandwidth of 20 MHz, 80 MHz, 10 MHz, or any other suitable bandwidth, both greater or smaller. The bandwidth of the subband may also be described or defined in terms of RBs. For example, the subband may include 50 RBs, 100 RBs, or any other suitable number of RBs, both greater or smaller. The first sidelink UE may transmit the synchronization communication such that the synchronization communication occupies the first portion of the plurality of RBs in the subband. In one aspect, the first portion of the subband includes 11 RBs. In some aspects, at least one of the RBs may be at or near an edge (e.g., lowest frequency or highest frequency) of the subband. For example, a first RB of the first portion of the subband may be a first RB of the subband. In other aspects, the first RB of the first portion may not the be first RB of the subband. For example, the subband may include one or more RBs on either side of the first portion of the subband, where the synchronization communication is transmitted in the first portion of the subband. In some instances, the first sidelink UE may utilize one or more components, such as the processor 702, the sidelink configuration module 708, the transceiver 710, the modem 712, and the one or more antennas 716, to perform the actions of block 810.

At block 820, the first sidelink UE transmits, to the second sidelink UE in the first slot and in the second portion of the subband of the shared frequency band, the discovery signal. The first sidelink UE may transmit the discovery signal such that the discovery signal comprises a waveform similar or identical to a PSSCH communication. Accordingly, the first sidelink UE may generate or prepare the discovery signal to include a DMRS pattern, MCS, RB allocation, TB size, and/or any other waveform parameter based on the respective parameters of a PSSCH waveform. In some aspects, the information carried in the discovery signal may be similar or identical to remaining minimum system information (RMSI). The discovery signal may include or indicate sidelink connection configuration information including one or more sidelink communication parameters. For example, the discovery signal may include or indicate at least one of a sidelink resource pool configuration, network service information associated with the first sidelink UE, and/or UE identity information associated with the first UE. Thus, the sidelink connection configuration may provide configuration information that can be used by a plurality of sidelink UEs to self-organize and coordinate transmissions, use of sidelink channel resources, detect and sync with other UEs, designate a coordinating or anchor UE, set up resource pools, and/or any other sidelink function.

In some aspects, transmitting the discovery signal includes transmitting a physical shared channel. For example, the physical shared channel may be similar or identical to the physical sidelink shared channel (PSSCH), in some aspects. In some aspects, the physical shared channel transmitted by the first sidelink UE may differ from a PSSCH in that control information (e.g., SCI) may be carried in the synchronization communication. For example, the MIB of the PBCH may carry the control information, as explained above. In some aspects, the first sidelink UE may transmit multiple repetitions of the discovery signal in a period of time. For example, the first sidelink UE may transmit multiple repetitions of the discovery signal within 80 ms intervals, or any other suitable interval. Accordingly, the second sidelink UE may perform soft decoding of the discovery signal based on the multiple repetitions.

The second portion of the subband of the shared frequency band may include one or more RBs forming a different portion of the subband than the RBs used to transmit the synchronization communication. As explained above, the second portion of the subband may be contiguous with the first portion of the subband. For example, the first sidelink UE may transmit the discovery signal in a second plurality of RBs that is contiguous with a first plurality of RBs used to transmit the synchronization communication. In some aspects, the number of RBs used by the first sidelink UE to transmit the discovery signal may depend on the subcarrier spacing of the shared frequency band. For example, if the shared frequency band is associated with a subcarrier spacing of 30 kHz, the first sidelink UE may transmit the discovery signal in a contiguous group of 36 RBs, and may transmit the synchronization communication in a contiguous group of 11 RBs. In another example, if the shared frequency band is associated with a subcarrier spacing of 15 kHz, the first sidelink UE may transmit the discovery signal in a contiguous group of 72 RBs. However, it will be understood that these values are examples, and that the first sidelink UE may transmit the discovery sidelink in any suitable number of RBs.

Further, as mentioned above, the first sidelink UE transmits the discovery signal in the first slot, which is the same slot used to transmit the synchronization communication. In some aspects, the first sidelink UE may transmit, and the second sidelink UE may receive, the discovery signal in the same 13 OFDM symbols used to transmit the synchronization communication, where the 14^{th} symbol is an idle symbol, or gap symbol. Accordingly, the discovery signal and the synchronization communication may be aligned in the time domain.

In some aspects, the first sidelink UE transmits the discovery signal such that it is quasi co-located (QCL) with the synchronization communication. Further, in some aspects, the first sidelink UE may transmit the discovery signal using an antenna port that is the same antenna portion used to transmit the synchronization communication. In some aspects, by transmitting the discovery signal such that it is aligned in time and QCL with the synchronization communication, and such that the discovery signal occupies a portion of the subband that is not occupied by the synchronization communication (e.g., S-SSB), the first sidelink UE may use network resources efficiently to send sidelink configuration information for sidelink coordination. Further, transmitting the discovery signal in this way may provide a flexible and inclusive discovery signaling scheme that can be used by devices associated with a variety of network service providers and equipment manufacturers.

For example, the method 800 may be used by UEs that have no network subscription, in some aspects. In some instances, the first sidelink UE may utilize one or more components, such as the processor 702, the sidelink configuration module 708, the transceiver 710, the modem 712, and the one or more antennas 716, to perform the actions of block 820.

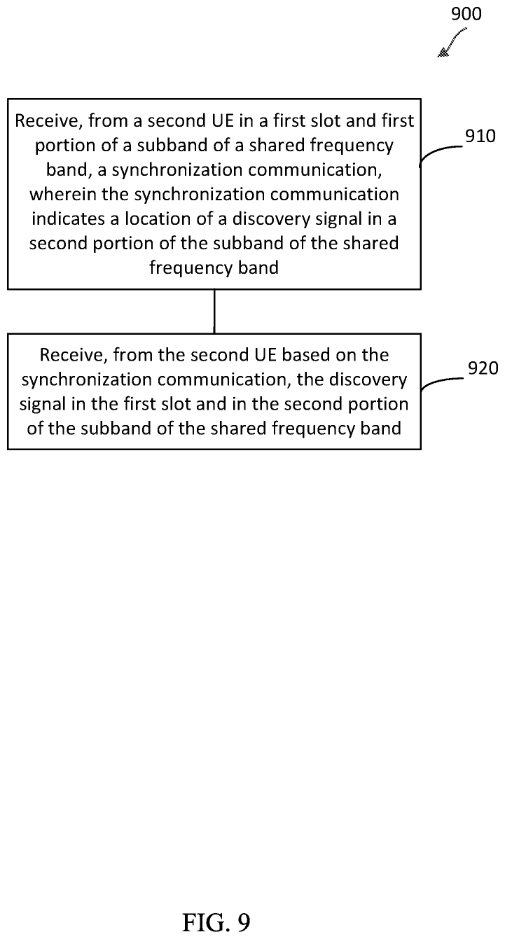

FIG. 9 is a flow diagram of a sidelink communication process 900 according to some aspects of the present disclosure. Aspects of the process 900 can be executed by a computing device (e.g., a processor, processing circuit, and/or other suitable component) of a wireless communication device or other suitable means for performing the steps. For example, a wireless communication device, such as the UEs 115, 215, and/or 700, may utilize one or more components, such as the processor 702, the memory 704, the sidelink configuration module 708, the transceiver 710, the modem 712, and the one or more antennas 716, to execute the steps of process 900. As illustrated, the process 900 includes a number of enumerated steps, but aspects of the process 900 may include additional steps before, after, and in between the enumerated steps. In some aspects, one or more of the enumerated steps may be omitted or performed in a different order.

At block 910, a first sidelink UE (e.g., UE 115j) receives, from a second sidelink UE in a first slot and a first portion of a subband of a shared frequency band, a synchronization communication. The synchronization communication may indicate a location of a discovery signal in a second portion of the subband of the shared frequency band. In some aspects, receiving the synchronization communication includes receiving a synchronization signal block (SSB), which includes a physical broadcast channel (PBCH), a primary synchronization signal (PSS), and a secondary synchronization signal (SSS). The SSB may be a sidelink SSB (S-SSB), for example. In some aspects, the first sidelink UE may receive a PBCH carrying a master information block (MIB). The MIB may have a structure similar or identical to the structure of the MIB 500 shown in FIG. 5. For example, the MIB may include a sidelink resource configuration that includes or indicates a modulation coding scheme, a resource block (RB) allocation, a transport block (TB) size, a demodulation reference signal (DMRS) pattern, and/or any other suitable parameter associated with the discovery signal. In some aspects, the first sidelink UE receives a PBCH including an indication of a first RB of the discovery signal and a size (e.g., in RBs) of the discovery signal. In some aspects, the first sidelink UE may also receive an indication of the location and/or periodicity of reference signals carried in the discovery signal. The synchronization information may include any suitable parameter or information to assist the first UE in detecting and decoding the discovery signal.

The first portion of the subband may include one or more contiguous RBs in the subband. In some aspects, the subband may be described as a channel having a bandwidth of 20 MHz, 80 MHz, 10 MHz, or any other suitable bandwidth, both greater or smaller. The bandwidth of the subband may also be described or defined in terms of RBs. For example, the subband may include 50 RBs, 100 RBs, or any other suitable number of RBs, both greater or smaller. The first sidelink UE may receive the synchronization communication in the first portion of the plurality of RBs in the subband. In one aspect, the first portion of the subband includes 11 RBs. In some aspects, at least one of the RBs may be at or near an edge (e.g., lowest frequency or highest frequency) of the subband. For example, a first RB of the first portion of the subband may be a first RB of the subband. In other aspects, the first RB of the first portion may not the be first RB of the subband. For example, the subband may include one or more RBs on either side of the first portion of the subband, where the synchronization communication is transmitted in the first portion of the subband. In some instances, the first sidelink UE may utilize one or more components, such as the processor 702, the sidelink configuration module 708, the transceiver 710, the modem 712, and the one or more antennas 716, to perform the actions of block 910.

At block 920, the first sidelink UE receives, from the second sidelink UE in the first slot and in the second portion of the subband of the shared frequency band, the discovery signal. The first sidelink UE may receive the discovery signal based on a waveform similar or identical to a PSSCH communication. Accordingly, the first sidelink UE may detect or decode the discovery signal based on a DMRS pattern, MCS, RB allocation, TB size, and/or any other waveform parameter based on the respective parameters of a PSSCH waveform. In some aspects, the information carried in the discovery signal may be similar or identical to remaining minimum system information (RMSI). The discovery signal may include or indicate one or more sidelink communication parameters. For example, the discovery signal may provide configuration information that can be used by a plurality of sidelink UEs to self-organize and coordinate transmissions, use of sidelink channel resources, detect and sync with other UEs, provide a coordinating or anchor UE, set up resource pools, and/or any other sidelink function.

In some aspects, receiving the discovery signal includes transmitting a physical shared channel. For example, the physical shared channel may be similar or identical to the physical sidelink shared channel (PSSCH), in some aspects. The physical shared channel received by the first sidelink UE may differ from a PSSCH in that control information (e.g., SCI) may be carried in the synchronization communication. For example, the MIB of the PBCH may carry the control information, as explained above. In some aspects, the first sidelink UE may receive multiple repetitions of the discovery signal in a period of time. For example, the first sidelink UE may receive multiple repetitions of the discovery signal within 80 ms intervals, or any other suitable interval. Accordingly, the first sidelink UE may perform soft decoding of the discovery signal based on the multiple repetitions.

The second portion of the subband of the shared frequency band may include one or more RBs forming a different portion of the subband than the RBs used to transmit the synchronization communication. As explained above, the second portion of the subband may be contiguous with the first portion of the subband. For example, the first sidelink UE may receive the discovery signal in a second plurality of RBs that is contiguous with a first plurality of RBs carrying the synchronization communication. In some aspects, the number of RBs carrying the discovery signal may depend on the subcarrier spacing of the shared frequency band. For example, if the shared frequency band is associated with a subcarrier spacing of 30 kHz, the first sidelink UE may receive the discovery signal in a contiguous group of 36 RBs, and may receive the synchronization communication in a different contiguous group of 11 RBs. In another example, if the shared frequency band is associated with a subcarrier spacing of 15 kHz, the first sidelink UE may receive the discovery signal in a contiguous group of 72 RBs. However, it will be understood that these values are examples, and that the first sidelink UE may receive the discovery sidelink in any suitable number of RBs.

Further, as mentioned above, the first sidelink UE receives the discovery signal in the first slot, which is the same slot in which the first sidelink UE receives the synchronization communication. In some aspects, the first sidelink UE may receive the discovery signal in the same 13 OFDM symbols used to transmit the synchronization communication, where the 14^{th} symbol is an idle symbol, or gap symbol. Accordingly, the discovery signal and the synchronization communication may be aligned in the time domain.

In some aspects, the first sidelink UE receives the discovery signal based on a quasi-co-located (QCL) relationship with the synchronization communication. Further, in some aspects, the first sidelink UE may receive the discovery signal using an antenna port that is the same antenna portion used to receive the synchronization communication. In some instances, the sidelink transmitting UE may utilize one or more components, such as the processor 702, the sidelink configuration module 708, the transceiver 710, the modem 712, and the one or more antennas 716, to perform the actions of block 920.

Further aspects of the present disclosure include the following:
1. A method performed by a first user equipment (UE), the method comprising:
   transmitting, to a second UE in a first slot and a first portion of a subband of a shared frequency band, a synchronization communication, wherein the synchronization communication indicates a location of a discovery signal in a second portion of the subband of the shared frequency band; and
   transmitting, to the second UE in the first slot and in the second portion of the subband of the shared frequency band, the discovery signal.
2. The method of clause 1, wherein the discovery signal indicates sidelink connection configuration information.
3. The method of clause 2, wherein the sidelink connection configuration information indicates at least one of:
   a sidelink resource pool configuration;
   network service information associated with the first UE; or
   UE identity information associated with the first UE.
4. The method of any of clauses 1-3, wherein the synchronization communication comprises a sidelink-synchronization signal block (S-SSB).
5. The method of any of clauses 1-4, wherein the synchronization communication indicates one or more control parameters associated with the discovery signal.
6. The method of clause 5, wherein the one or more control parameters indicates at least one of:
   a modulation and coding scheme (MCS) of the discovery signal;
   a resource block (RB) allocation of the discovery signal;
   a transport block (TB) size of the discovery signal; or
   a demodulation reference signal (DMRS) pattern of the discovery signal.
7. The method of any of clauses 1-6, wherein the transmitting the discovery signal comprises transmitting two or more repetitions of the discovery signal within a period of time.
8. The method of any of clauses 1-7, wherein the transmitting the discovery signal comprises transmitting the discovery signal based on a physical sidelink shared channel (PSSCH) waveform.
9. The method of any of clauses 1-8, wherein the second portion of the subband comprises a plurality of resource blocks (RBs), wherein the plurality of RBs is contiguous with RBs of the synchronization communication.
10. The method of any of clauses 1-9, wherein the transmitting the discovery signal comprises transmitting the discovery signal such that the discovery signal is quasi co-located (QCL) with the synchronization communication.
11. A method performed by a first user equipment (UE), the method comprising:
   receiving, from a second UE in a first slot and first portion of a subband of a shared frequency band, a synchronization communication, wherein the synchronization communication indicates a location of a discovery signal in a second portion of the subband of the shared frequency band; and
   receiving, from the second UE based on the synchronization communication, the discovery signal in the first slot and in the second portion of the subband of the shared frequency band.
12. The method of clause 11,
   wherein the discovery signal comprises sidelink configuration information indicating at least one of:
      a sidelink resource pool configuration;
      network service information associated with the second UE; or
      UE identity information associated with the second UE.
13. The method of clause 12, further comprising:
   transmitting, based on the sidelink configuration information, a sidelink communication to the second UE.
14. The method of any of clauses 12-13, further comprising:
   transmitting, based on the sidelink configuration information, an uplink (UL) communication to a base station (BS) via the second UE.
15. The method of any of clauses 11-14,
   wherein the synchronization communication comprises a master information block (MIB),
   wherein the MIB indicates one or more control parameters associated with the discovery signal, and
   wherein the receiving the discovery signal comprises receiving the discovery signal based on the one or more control parameters.
16. The method of clause 15, wherein the one or more control parameters indicates at least one of:
   a modulation and coding scheme (MCS) of the discovery signal;
   a resource block (RB) allocation of the discovery signal;
   a transport block (TB) size of the discovery signal; or
   a demodulation reference signal (DMRS) pattern of the discovery signal.
17. The method of any of clauses 11-16,
   wherein the receiving the discovery signal comprises receiving two or more repetitions of the discovery signal within a period of time, and
   wherein the method further comprises decoding the discovery signal based on the two or more repetitions of the discovery signal.
18. A first user equipment (UE), comprising:
   a transceiver; and
   a processor in communication with the transceiver, wherein the processor is configured to cause the transceiver to:
      transmit, to a second UE in a first slot and a first portion of a subband of a shared frequency band, a synchronization communication, wherein the synchronization communication indicates a location of a discovery signal in a second portion of the subband of the shared frequency band; and
      transmit, to the second UE in the first slot and in the second portion of the subband of the shared frequency band, the discovery signal.
19. The first UE of clause 18, wherein the discovery signal indicates sidelink connection configuration information.
20. The first UE of clause 19, wherein the sidelink connection configuration information indicates at least one of:
   a sidelink resource pool configuration;
   network service information associated with the first UE; or
   UE identity information associated with the first UE.
21. The first UE of any of clauses 18-20, wherein the synchronization communication comprises a sidelink-synchronization signal block (S-SSB).
22. The first UE of any of clauses 18-21, wherein the synchronization communication indicates one or more control parameters associated with the discovery signal.
23. The first UE of clause 22, wherein the one or more control parameters indicates at least one of:
   a modulation and coding scheme (MCS) of the discovery signal;
   a resource block (RB) allocation of the discovery signal;
   a transport block (TB) size of the discovery signal; or
   a demodulation reference signal (DMRS) pattern of the discovery signal.
24. The first UE of any of clauses 18-23, wherein the processor configured to cause the transceiver to transmit the discovery signal comprises the processor configured to cause the transceiver to transmit the discovery signal based on a physical sidelink shared channel (PSSCH) waveform.
25. The first UE of any of clauses 18-24, wherein the processor configured to cause the transceiver to transmit the discovery signal comprises the processor configured to cause the transceiver to transmit the discovery signal such that the discovery signal is quasi co-located (QCL) with the synchronization communication.
26. A first user equipment (UE), comprising:
   a transceiver; and
   a processor in communication with the transceiver, wherein the processor is configured to cause the transceiver to:
      receive, from a second UE in a first slot and first portion of a subband of a shared frequency band, a synchronization communication, wherein the synchronization communication indicates a location of a discovery signal in a second portion of the subband of the shared frequency band; and
      receive, from the second UE based on the synchronization communication, the discovery signal in the first slot and in the second portion of the subband of the shared frequency band.
27. The first UE of clause 26,
   wherein the discovery signal comprises sidelink configuration information indicating at least one of:
      a sidelink resource pool configuration;
      network service information associated with the second UE; or
      UE identity information associated with the second UE.
28. The first UE of any of clauses 26 and 27,
   wherein the synchronization communication comprises a master information block (MIB),
   wherein the MIB indicates one or more control parameters associated with the discovery signal, and
   wherein the processor configured to cause the transceiver to receive the discovery signal comprises the processor configured to cause the transceiver to receive the discovery signal based on the one or more control parameters.
29. The first UE of clause 28, wherein the one or more control parameters indicates at least one of:
   a modulation and coding scheme (MCS) of the discovery signal;
   a resource block (RB) allocation of the discovery signal;
   a transport block (TB) size of the discovery signal; or
   a demodulation reference signal (DMRS) pattern of the discovery signal.
30. The first UE of any of clauses 26-30,
   wherein the processor configured to cause the transceiver to receive the discovery signal comprises the processor configured to cause the transceiver to receive two or more repetitions of the discovery signal within a period of time, and
   wherein the processor is further configured to decode the discovery signal based on the two or more repetitions of the discovery signal.
31. A non-transitory computer-readable medium having program code recorded thereon, wherein the program code comprises:
   code for causing a first user equipment (UE) to transmit, to a second UE in a first slot and a first portion of a subband of a shared frequency band, a synchronization communication, wherein the synchronization communication indicates a location of a discovery signal in a second portion of the subband of the shared frequency band; and
   code for causing the first UE to transmit, to the second UE in the first slot and in the second portion of the subband of the shared frequency band, the discovery signal.
32. The non-transitory computer-readable medium of claim 31, wherein the discovery signal indicates sidelink connection configuration information.
33. The non-transitory computer-readable medium of claim 32, wherein the sidelink connection configuration information indicates at least one of:
   a sidelink resource pool configuration;
   network service information associated with the first UE; or
   UE identity information associated with the first UE.
34. The non-transitory computer-readable medium of any of claims 31-33, wherein the synchronization communication comprises a sidelink-synchronization signal block (S-SSB).
35. The non-transitory computer-readable medium of any of claims 31-34, wherein the synchronization communication indicates one or more control parameters associated with the discovery signal.
36. The non-transitory computer-readable medium of claim 35, wherein the one or more control parameters indicates at least one of:
   a modulation and coding scheme (MCS) of the discovery signal;
   a resource block (RB) allocation of the discovery signal;
   a transport block (TB) size of the discovery signal; or
   a demodulation reference signal (DMRS) pattern of the discovery signal.
37. The non-transitory computer-readable medium of any of claims 31-36, wherein the code for causing the first UE to transmit the discovery signal comprises code for causing the first UE to transmit two or more repetitions of the discovery signal within a period of time.
38. The non-transitory computer-readable medium of any of claims 31-37, wherein the code for causing the first UE to transmit the discovery signal comprises code for causing the first UE to transmit the discovery signal based on a physical sidelink shared channel (PSSCH) waveform.
39. The non-transitory computer-readable medium of any of claims 31-38, wherein the second portion of the subband comprises a plurality of resource blocks (RBs), wherein the plurality of RBs is contiguous with RBs of the synchronization communication.
40. The non-transitory computer-readable medium of any of claims 31-39, wherein the code for causing the first UE to transmit the discovery signal comprises code for causing the first UE to transmit the discovery signal such that the discovery signal is quasi co-located (QCL) with the synchronization communication.
41. A non-transitory computer-readable medium having program code recorded thereon, wherein the program code comprises:
   receiving, from a second UE in a first slot and first portion of a subband of a shared frequency band, a synchronization communication, wherein the synchronization communication indicates a location of a discovery signal in a second portion of the subband of the shared frequency band; and
   receiving, from the second UE based on the synchronization communication, the discovery signal in the first slot and in the second portion of the subband of the shared frequency band.
42. The non-transitory computer-readable medium of claim 41,
   wherein the discovery signal comprises sidelink configuration information indicating at least one of:
      a sidelink resource pool configuration;
      network service information associated with the second UE; or
      UE identity information associated with the second UE.
43. The non-transitory computer-readable medium of claim 42, further comprising:
   transmitting, based on the sidelink configuration information, a sidelink communication to the second UE.
44. The non-transitory computer-readable medium of any of claims 42-43, wherein the program code further comprises:
   code for causing the first UE to transmit, based on the sidelink configuration information, an uplink (UL) communication to a base station (BS) via the second UE.
45. The non-transitory computer-readable medium of any of claims 41-44,
   wherein the synchronization communication comprises a master information block (MIB),
   wherein the MIB indicates one or more control parameters associated with the discovery signal, and
   wherein the code for causing the first UE to receive the discovery signal comprises code for causing the first UE to receive the discovery signal based on the one or more control parameters.
46. The non-transitory computer-readable medium of claim 45, wherein the one or more control parameters indicates at least one of:
   a modulation and coding scheme (MCS) of the discovery signal;
   a resource block (RB) allocation of the discovery signal;
   a transport block (TB) size of the discovery signal; or
   a demodulation reference signal (DMRS) pattern of the discovery signal.
47. The non-transitory computer-readable medium of any of claims 41-46,
   wherein the code for causing the first UE to receive the discovery signal comprises code for causing the first UE to receive two or more repetitions of the discovery signal within a period of time, and
   wherein the program code further comprises code for causing the first UE to decode the discovery signal based on the two or more repetitions of the discovery signal.
48. A first user equipment (UE), comprising:
   means for transmitting, to a second UE in a first slot and a first portion of a subband of a shared frequency band, a synchronization communication, wherein the synchronization communication indicates a location of a discovery signal in a second portion of the subband of the shared frequency band; and
   means for transmitting, to the second UE in the first slot and in the second portion of the subband of the shared frequency band, the discovery signal.
49. The first UE of clause 48, wherein the discovery signal indicates sidelink connection configuration information.
50. The first UE of clause 49, wherein the sidelink connection configuration information indicates at least one of:
   a sidelink resource pool configuration;
   network service information associated with the first UE; or
   UE identity information associated with the first UE.
51. The first UE of any of clauses 48-50, wherein the synchronization communication comprises a sidelink-synchronization signal block (S-SSB).
52. The first UE of any of clauses 48-51, wherein the synchronization communication indicates one or more control parameters associated with the discovery signal.
53. The first UE of clause 52, wherein the one or more control parameters indicates at least one of:
   a modulation and coding scheme (MCS) of the discovery signal;
   a resource block (RB) allocation of the discovery signal;
   a transport block (TB) size of the discovery signal; or
   a demodulation reference signal (DMRS) pattern of the discovery signal.
54. The first UE of any of clauses 48-53, wherein the means for transmitting the discovery signal comprises means for transmitting two or more repetitions of the discovery signal within a period of time.
55. The first UE of any of clauses 48-54, wherein the means for transmitting the discovery signal comprises means for transmitting the discovery signal based on a physical sidelink shared channel (PSSCH) waveform.
56. The first UE of any of clauses 48-55, wherein the second portion of the subband comprises a plurality of resource blocks (RBs), wherein the plurality of RBs is contiguous with RBs of the synchronization communication.
57. The first UE of any of clauses 48-56, wherein the means for transmitting the discovery signal comprises means for transmitting the discovery signal such that the discovery signal is quasi co-located (QCL) with the synchronization communication.
58. A first user equipment (UE), comprising:
   means for receiving, from a second UE in a first slot and first portion of a subband of a shared frequency band, a synchronization communication, wherein the synchronization communication indicates a location of a discovery signal in a second portion of the subband of the shared frequency band; and
   means for receiving, from the second UE based on the synchronization communication, the discovery signal in the first slot and in the second portion of the subband of the shared frequency band.
59. The first UE of clause 58,
   wherein the discovery signal comprises sidelink configuration information indicating at least one of:
      a sidelink resource pool configuration;
      network service information associated with the second UE; or
      UE identity information associated with the second UE.
60. The first UE of clause 59, further comprising:
   means for transmitting, based on the sidelink configuration information, a sidelink communication to the second UE.
61. The first UE of any of clauses 59-60, further comprising:
   means for transmitting, based on the sidelink configuration information, an uplink (UL) communication to a base station (BS) via the second UE.
62. The first UE of any of clauses 58-61,
   wherein the synchronization communication comprises a master information block (MIB),
   wherein the MIB indicates one or more control parameters associated with the discovery signal, and
   wherein the means for receiving the discovery signal comprises means for receiving the discovery signal based on the one or more control parameters.
63. The first UE of clause 62, wherein the one or more control parameters indicates at least one of:
   a modulation and coding scheme (MCS) of the discovery signal;
   a resource block (RB) allocation of the discovery signal;
   a transport block (TB) size of the discovery signal; or
   a demodulation reference signal (DMRS) pattern of the discovery signal.
64. The first UE of any of clauses 58-63,
   wherein the means for receiving the discovery signal comprises means for receiving two or more repetitions of the discovery signal within a period of time, and
   wherein the method further comprises means for decoding the discovery signal based on the two or more repetitions of the discovery signal.

Information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of [at least one of A, B, or C] means A or B or C or AB or AC or BC or ABC (i.e., A and B and C).

As those of some skill in this art will by now appreciate and depending on the particular application at hand, many modifications, substitutions and variations can be made in and to the materials, apparatus, configurations and methods of use of the devices of the present disclosure without departing from the spirit and scope thereof. In light of this, the scope of the present disclosure should not be limited to that of the particular embodiments illustrated and described herein, as they are merely by way of some examples thereof, but rather, should be fully commensurate with that of the claims appended hereafter and their functional equivalents.

What is claimed is:

1. A method performed by a first user equipment (UE), the method comprising:
transmitting, to a second UE in a first slot and a first portion of a subband of a shared frequency band, a synchronization communication, wherein the synchronization communication indicates a location of a discovery signal in a second portion of the subband of the shared frequency band; and
transmitting, to the second UE in the first slot and in the second portion of the subband of the shared frequency band, the discovery signal.

2. The method of claim 1, wherein the discovery signal indicates sidelink connection configuration information.

3. The method of claim 2, wherein the sidelink connection configuration information indicates at least one of:
a sidelink resource pool configuration;
network service information associated with the first UE; or
UE identity information associated with the first UE.

4. The method of claim 1, wherein the synchronization communication comprises a sidelink-synchronization signal block (S-SSB).

5. The method of claim 1, wherein the synchronization communication indicates one or more control parameters associated with the discovery signal.

6. The method of claim 5, wherein the one or more control parameters indicates at least one of:
a modulation and coding scheme (MCS) of the discovery signal;
a resource block (RB) allocation of the discovery signal;
a transport block (TB) size of the discovery signal; or
a demodulation reference signal (DMRS) pattern of the discovery signal.

7. The method of claim 1, wherein the transmitting the discovery signal comprises transmitting two or more repetitions of the discovery signal within a period of time.

8. The method of claim 1, wherein the transmitting the discovery signal comprises transmitting the discovery signal based on a physical sidelink shared channel (PSSCH) waveform.

9. The method of claim 1, wherein the second portion of the subband comprises a plurality of resource blocks (RBs), wherein the plurality of RBs is contiguous with RBs of the synchronization communication.

10. The method of claim 1, wherein the transmitting the discovery signal comprises transmitting the discovery signal such that the discovery signal is quasi co-located (QCL) with the synchronization communication.

11. A method performed by a first user equipment (UE), the method comprising:
receiving, from a second UE in a first slot and first portion of a subband of a shared frequency band, a synchronization communication, wherein the synchronization communication indicates a location of a discovery signal in a second portion of the subband of the shared frequency band; and
receiving, from the second UE based on the synchronization communication, the discovery signal in the first slot and in the second portion of the subband of the shared frequency band.

12. The method of claim 11,
wherein the discovery signal comprises sidelink configuration information indicating at least one of:
a sidelink resource pool configuration;
network service information associated with the second UE; or
UE identity information associated with the second UE.

13. The method of claim 12, further comprising:
transmitting, based on the sidelink configuration information, a sidelink communication to the second UE.

14. The method of claim 12, further comprising:
transmitting, based on the sidelink configuration information, an uplink (UL) communication to a base station (BS) via the second UE.

15. The method of claim 11,
wherein the synchronization communication comprises a master information block (MIB),
wherein the MIB indicates one or more control parameters associated with the discovery signal, and
wherein the receiving the discovery signal comprises receiving the discovery signal based on the one or more control parameters.

16. The method of claim 15, wherein the one or more control parameters indicates at least one of:
a modulation and coding scheme (MCS) of the discovery signal;
a resource block (RB) allocation of the discovery signal;
a transport block (TB) size of the discovery signal; or
a demodulation reference signal (DMRS) pattern of the discovery signal.

17. The method of claim 11,
wherein the receiving the discovery signal comprises receiving two or more repetitions of the discovery signal within a period of time, and
wherein the method further comprises decoding the discovery signal based on the two or more repetitions of the discovery signal.

18. A first user equipment (UE), comprising:
a transceiver; and
a processor in communication with the transceiver, wherein the processor is configured to cause the transceiver to:
transmit, to a second UE in a first slot and a first portion of a subband of a shared frequency band, a synchronization communication, wherein the synchronization communication indicates a location of a discovery signal in a second portion of the subband of the shared frequency band; and
transmit, to the second UE in the first slot and in the second portion of the subband of the shared frequency band, the discovery signal.

19. The first UE of claim 18, wherein the discovery signal indicates sidelink connection configuration information.

20. The first UE of claim 19, wherein the sidelink connection configuration information indicates at least one of:
a sidelink resource pool configuration;
network service information associated with the first UE; or
UE identity information associated with the first UE.

21. The first UE of claim 18, wherein the synchronization communication comprises a sidelink-synchronization signal block (S-SSB).

22. The first UE of claim 18, wherein the synchronization communication indicates one or more control parameters associated with the discovery signal.

23. The first UE of claim 22, wherein the one or more control parameters indicates at least one of:
a modulation and coding scheme (MCS) of the discovery signal;
a resource block (RB) allocation of the discovery signal;
a transport block (TB) size of the discovery signal; or
a demodulation reference signal (DMRS) pattern of the discovery signal.

24. The first UE of claim 18, wherein the processor configured to cause the transceiver to transmit the discovery signal comprises the processor configured to cause the transceiver to transmit the discovery signal based on a physical sidelink shared channel (PSSCH) waveform.

25. The first UE of claim 18, wherein the processor configured to cause the transceiver to transmit the discovery signal comprises the processor configured to cause the transceiver to transmit the discovery signal such that the discovery signal is quasi co-located (QCL) with the synchronization communication.

26. A first user equipment (UE), comprising:
a transceiver; and
a processor in communication with the transceiver, wherein the processor is configured to cause the transceiver to:
receive, from a second UE in a first slot and first portion of a subband of a shared frequency band, a synchronization communication, wherein the synchronization communication indicates a location of a discovery signal in a second portion of the subband of the shared frequency band; and
receive, from the second UE based on the synchronization communication, the discovery signal in the first slot and in the second portion of the subband of the shared frequency band.

27. The first UE of claim 26,
wherein the discovery signal comprises sidelink configuration information indicating at least one of:
a sidelink resource pool configuration;
network service information associated with the second UE; or
UE identity information associated with the second UE.

28. The first UE of claim 26,
wherein the synchronization communication comprises a master information block (MIB),
wherein the MIB indicates one or more control parameters associated with the discovery signal, and
wherein the processor configured to cause the transceiver to receive the discovery signal comprises the processor configured to cause the transceiver to receive the discovery signal based on the one or more control parameters.

29. The first UE of claim 28, wherein the one or more control parameters indicates at least one of:
a modulation and coding scheme (MCS) of the discovery signal;
a resource block (RB) allocation of the discovery signal;
a transport block (TB) size of the discovery signal; or
a demodulation reference signal (DMRS) pattern of the discovery signal.

30. The first UE of claim 26,
wherein the processor configured to cause the transceiver to receive the discovery signal comprises the processor configured to cause the transceiver to receive two or more repetitions of the discovery signal within a period of time, and
wherein the processor is further configured to decode the discovery signal based on the two or more repetitions of the discovery signal.
